(19) United States
(12) Patent Application Publication
(10) Pub. No.: US 2011/0281822 A1
(43) Pub. Date: Nov. 17, 2011

(54) AROMATIC COMPOUNDS HAVING SPHINGOSINE-1-PHOSPHONATE (S1P) RECEPTOR ACTIVITY

(75) Inventors: Janet A. Takeuchi, Anaheim, CA (US); Ken Chow, Newport Coast, CA (US); Ling Li, Irvine, CA (US); Xiaoxia Liu, Lake Forest, CA (US)

(73) Assignee: ALLERGAN, INC., Irvine, CA (US)

(21) Appl. No.: 13/105,590

(22) Filed: May 11, 2011

Related U.S. Application Data
(60) Provisional application No. 61/334,937, filed on May 14, 2010.

(51) Int. Cl. A61K 31/675; A61K 31/661; A61P 27/02; A61P 27/06; A61P 9/10; A61P 29/00; A61P 31/00; A61P 35/04; A61P 9/00; A61P 37/04; A61P 1/00; A61P 5/14; A61P 25/00; A61P 17/06; A61P 1/04; A61P 17/00; A61P 37/08; A61P 11/06; A61P 11/00; A61P 17/02; A61Q 19/08; A61P 19/00; A61P 19/10; A61P 21/00; A61P 19/02; A61P 21/04; A61P 25/28; C07F 9/141

(52) U.S. Cl. 514/89; 546/22; 558/166; 548/413; 514/114; 514/91

(57) ABSTRACT

Novel aromatic compounds which are useful as sphingosine-1-phosphate modulators and useful for treating a wide variety of disorders associated with modulation of sphingosine-1-phosphate receptors.

### RELATED APPLICATIONS

This application is based on, and claims priority under 35 U.S.C. §120 to U.S. Provisional Application No. 61/334,937, filed on May 14, 2010, and which is incorporated herein by reference.

### FIELD OF THE INVENTION

The present invention relates to aromatic compounds processes for preparing them, pharmaceutical compositions containing them and their use as pharmaceuticals as modulators of sphingosine-1-phosphate receptors. The invention relates specifically to the use of these compounds and their pharmaceutical compositions to treat disorders associated with sphingosine-1-phosphate (S1P) receptor modulation.

### BACKGROUND OF THE INVENTION

Sphingosine-1 phosphate is stored in relatively high concentrations in human platelets, which lack the enzymes responsible for its catabolism, and it is released into the blood stream upon activation of physiological stimuli, such as growth factors, cytokines, and receptor agonists and antigens. It may also have a critical role in platelet aggregation and thrombosis and could aggravate cardiovascular diseases. On the other hand the relatively high concentration of the metabolite in high-density lipoproteins (HDL) may have beneficial implications for atherogenesis. For example, there are recent suggestions that sphingosine-1-phosphate, together with other lysolipids such as sphingosylphosphorylcholine and lysosulfatide, are responsible for the beneficial clinical effects of HDL by stimulating the production of the potent antiatherogenic signaling molecule nitric oxide by the vascular endothelium. In addition, like lysophosphatidic acid, it is a marker for certain types of cancer, and there is evidence that its role in cell division or proliferation may have an influence on the development of cancers. These are currently topics that are attracting great interest amongst medical researchers, and the potential for therapeutic intervention in sphingosine-1-phosphate metabolism is under active investigation.

Published International Patent Application No. WO 2008030843 describes heterocyclic aminophosphonates and oxyphosphonates having sphingosine-1-phosphate receptor biological activity.

Published International Patent Application No. WO 2008030838 describes heteroaromatic derivatives as sphingosine-1-phosphate receptor agonists and theft preparation and use in the treatment of diseases.

Published International Patent Application No. WO 2008141013 describes Sphingosine-1-phosphate 3 receptor inhibitors for the treatment of pain.

Published International Patent Application No. WO 9202513 describes the preparation of diphenylazines as antithrombotics vasodilators, antihypertensives, and antiinflammatories.

Granted U.S. Pat. No. 7,728,014 discloses heteroaromatic compounds having biological activity at the sphingosine-1-phosphate 3 receptor.

### SUMMARY OF THE INVENTION

We have now discovered a group of novel compounds which are potent and selective sphingosine-1-phosphate modulators. As such, the compounds described herein are useful in treating a wide variety of disorders associated with modulation of sphingosine-1-phosphate receptors. The term “modulator” as used herein, includes but is not limited to: receptor agonist, antagonist, inverse agonist, inverse antagonist, partial agonist, partial antagonist.

This invention describes compounds of Formula I, which have sphingosine-1-phosphate receptor biological activity. The compounds in accordance with the present invention are thus of use in medicine, for example in the treatment of humans with diseases and conditions that are alleviated by S1P modulation.

In one aspect, the invention provides a compound having Formula I or a pharmaceutically acceptable salt thereof or stereoisomeric forms thereof, or the geometrical isomers, enantiomers, diastereoisomers, tautomers, zwitterions and pharmaceutically acceptable salts thereof:

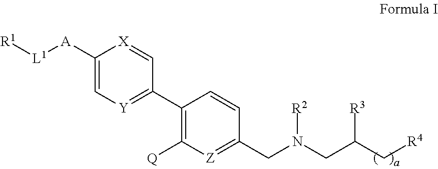

wherein:

R¹ is Me, CF₃ or aryl;

R² is H, C₁₋₁₀ alkyl, or together with R³ forms a 5 or 6 membered heterocycle ring;

R³ is H, C₁₋₁₀ alkyl, or together with R² forms a 5 or 6 membered heterocycle ring;

R⁴ is OPO₃H₂, carboxylic acid, C₁₋₆ alkyl, —S(O)₂H, —P(O)(OH)(OR¹⁰), —P(O)(H)OH or OR⁹;

X is CR⁵ or N;

Y is CR⁶ or N;

Z is CR⁷ or N;

A is O, CH₂ or NR⁸;

L¹ is C₂₋₁₀ alkylene;

R⁵ is H, C₁₋₁₀ alkyl, C₂₋₆ alkenyl or C₃₋₁₀ cycloalkyl;

R⁶ is H, C₁₋₁₀ alkyl, C₂₋₆ alkenyl or C₃₋₁₀ cycloalkyl;

R⁷ is H, C₁₋₁₀ alkyl, C₂₋₆ alkenyl or C₃₋₁₀ cycloalkyl;

R⁸ is H, C₃₋₁₀ cycloalkyl or C₁₋₆ alkyl;

R⁹ is H or C₁₋₁₀ alkyl;

R¹⁰ is H or C₁₋₁₀ alkyl;

Q is C₃₋₁₀ cycloalkyl, heterocycle or aryl; and

a is 0, 1, 2, 3 or 4.

The term “alkyl”, as used herein, refers to saturated, monovalent hydrocarbon moieties having linear or branched moieties or combinations thereof and containing 1 to 10 carbon atoms. One methylene (—CH₂—) group, of the alkyl can be replaced by oxygen, sulfur, sulfoxide, nitrogen, carbonyl, carboxyl, sulfonyl, or by a divalent C₃₋₁₀ cycloalkyl. Alkyl groups can be substituted by halogen, hydroxyl, cycloalkyl, amino, heterocycles, carboxylic acid, phosphonic acid groups, sulphonic acid groups, phosphoric acid. Usually, in the present case, alkyl groups are methyl, n-butyl, n-propyl, hexafluoropropyl, trifluoromethyl.

The term “alkylene”, as used herein, refers to saturated, divalent hydrocarbon moieties having linear or branched moieties or combinations thereof and containing 2 to 10 carbon atoms. One methylene (—CH₂—) group, of the alkylene can be replaced by oxygen, sulfur, sulfoxide, nitrogen, carbonyl, carboxyl, sulfonyl, or by a divalent C₃₋₁₀ cycloalkyl. Alkylene groups can be substituted by halogen, hydroxyl, cycloalkyl, amino, heterocycles, carboxylic acid, phosphonic acid groups, sulphonic acid groups, phosphoric acid. Usually, in the present case, alkylene groups are ethylene, n-butylene, n-propylene, hexafluoropropylene.

The term “cycloalkyl”, as used herein, refers to a monovalent or divalent group of 3 to 10 carbon atoms, preferably 3 to 5 carbon atoms derived from a saturated cyclic hydrocarbon. Cycloalkyl groups can be monocyclic or polycyclic. Cycloalkyl can be substituted by C₁₋₆ alkyl groups or halogens.

The term “alkenyl”, as used herein, refers to a monovalent or divalent hydrocarbon radical having 2 to 6 carbon atoms, derived from a saturated alkyl, having at least one double bond. C₂₋₆ alkenyl can be in the E or Z configuration. Alkenyl groups can be substituted by C₁₋₆ alkyl, as defined above, or by halogen.

The term “halogen”, as used herein, refers to an atom of chlorine, bromine, fluorine, iodine. Usually, in the present case, halogen group is fluoro.

The term “heterocycle” as used herein, refers to a 3 to 10 membered ring, which can be aromatic or non-aromatic, saturated or non-saturated, containing at least one heteroatom selected form O or N or S or combinations of at least two thereof, interrupting the carbocyclic ring structure. The heterocyclic ring can be saturated or non-saturated. The heterocyclic ring can be interrupted by a C═O; the S heteroatom can be oxidized. Heterocycles can be monocyclic or polycyclic. Heterocyclic ring moieties can be substituted by hydroxyl, C₁₋₆ alkyl or halogens. Usually, in the present case, heterocyclic groups are pyridine, thiopene, furan, thiazol, oxazol, pyrroline, 5-fluoro-thiophen-2-yl.

The term “aryl” as used herein, refers to an organic moiety derived from an aromatic hydrocarbon consisting of a ring containing 6 to 10 carbon atoms by removal of one hydrogen, which can be substituted by halogen atoms, —OC₁₋₃ alkyl, C₁₋₃ alkyl, nitrile, C(O)C₁₋₃ alkyl, amino or hydroxyl groups. Usually, in the present case, aryl is phenyl, 3-fluorophenyl, 4-fluorophenyl, 3-hydroxylphenyl.

The term “hydroxyl” as used herein, represents a group of formula “—OH”.

The formula “H”, as used herein, represents a hydrogen atom.

The formula “O”, as used herein, represents an oxygen atom.

The formula “N”, as used herein, represents a nitrogen atom.

The formula “S”, as used herein, represents a sulfur atom.

The term “nitrile”, as used herein, represents a group of formula “—CN”.

The term “sulfoxide” as used herein, represents a group of formula “—S(O)”.

The term “carbonyl” as used herein, represents a group of formula “—C(O)”.

The term “carboxyl” as used herein, represents a group of formula “—(CO)O—”.

The term “sulfonyl” as used herein, represents a group of formula —SO₂”.

The term “carboxylic acid” as used herein, represents a group of formula “—COOH”.

The term “CF₃” as used herein, represents a trifluoromethyl group.

The term “amino” as used herein, represents a group of formula “—NH₂” or “—NH(C₁₋₆ alkyl)” or “—N(C₁₋₆ alkyl)(C₁₋₆ alkyl)”.

The term “phosphonic acid” as used herein, represents a group of formula “—P(O)(OH)₂”.

The term “sulphonic acid” as used herein, represents a group of formula “—SO₂(OH)”.

The term “phosphoric acid” as used herein, represents a group of formula “—OP(O)(OH)₂”.

The term “Me”, as used herein represents a methyl group.

Generally, R¹ is selected from Me, CF₃ or aryl. Usually R¹ is Me, CF₃, or phenyl.

Generally, R² is selected from H, C₁₋₁₀ alkyl, or together with R³ forms a 5 or 6 membered heterocycle ring. Usually R² is H or form together with R³ a pyrrolidine ring.

Generally, R³ is selected from H, C₁₋₁₀ alkyl, or together with R² forms a 5 or 6 membered heterocycle ring. Usually R³ is H or form together with R² a pyrrolidine ring.

Generally, R⁴ is selected from OPO₃H₂, carboxylic acid, C₁₋₆ alkyl, —S(O)₂H, —P(O)(OH)(OR¹⁰), —P(O)(H)OH or OR⁹. Usually, R⁴ is P(O)(OH)(OR¹⁰).

Generally, X is CR⁵ or N. Usually X is CH, N or C—C₁₋₆ alkyl.

Generally, Y is CR⁶ or N. Usually, Y is CH or N.

Generally, Z is CR⁷ or N. Usually, Z is CH or N.

Generally, A is O, CH₂ or NR⁸. Usually, A is O or CH₂.

Generally, L¹ is C₂₋₁₀ alkylene. Usually, L¹ is ethylene, n-butylene, n-propylene, hexafluoropropylene.

Generally, R⁵ is H, C₁₋₁₀ alkyl, C₂₋₆ alkenyl or C₃₋₁₀ cycloalkyl. Usually, R⁵ is H or propyl.

Generally, R⁶ is H, C₁₋₁₀ alkyl, C₂₋₆ alkenyl or C₃₋₁₀ cycloalkyl. Usually, R⁶ is H.

Generally, R⁷ is H, C₁₋₁₀ alkyl, C₂₋₆ alkenyl or C₃₋₁₀ cycloalkyl. Usually, R⁷ is H.

Generally, R⁸ is H, C₃₋₁₀ cycloalkyl or C₁₋₆ alkyl.

Generally, R⁹ is H or C₁₋₁₀ alkyl.

Generally, R¹⁰ is H or C₁₋₁₀ alkyl. Usually, R¹⁰ is H or ethyl.

Generally, Q is C₃₋₁₀ cycloalkyl, heterocycle or aryl. Usually, Q is phenyl, pyridinyl, thiopene, oxazole, thiazole, 3-fluorophenyl, 4-fluorophenyl, 3-hydroxylphenyl, 5-fluoro-thiophen-2-yl.

Generally, a is 0, 1, 2, 3 or 4. Usually, a is 0 or 1.

In one embodiment of the invention

R¹ is Me, CF₃, phenyl; and

R² is H, or together with R³ forms a 5 membered heterocycle ring; and

R³ is H, or together with R² forms a 5 membered heterocycle ring; and

R⁴ is —P(O)(OH)(OR¹⁰); and

X is CR⁵ or N; and

Y is CR⁶ or N; and

Z is CR⁷ or N; and

A is O or CH₂; and

L¹ is C₂₋₅ alkylene; and

R⁵ is H or C₁₋₆ alkyl; and

R⁶ is H; and

R⁷ is H; and

R¹⁰ is H or C₁₋₆ alkyl; and

Q is heterocycle or aryl; and

a is 0 or 1.

In a preferred embodiment of the invention

R¹ is Me or phenyl; and

R² is H; and

R³ is H; and

R⁴ is —P(O)(OH)(OR¹⁰); and

X is CR⁵; and

Y is CR⁶ or N; and

Z is N; and

A is CH₂; and

L¹ is C₂₋₅ alkylene; and

R⁵ is H or C₁₋₆ alkyl; and

R⁶ is H; and

R¹⁰ is H; and

Q is heterocycle or aryl; and

a is 1.

Compounds of the invention are:
(3-{[6-(5-Hexyl-pyridin-2-yl)-biphenyl-3-ylmethyl]-amino}-propyl)-phosphonic acid;
(3-{[6-(6-Hexyl-pyridin-3-yl)-biphenyl-3-ylmethyl]-amino}-propyl)-phosphonic acid;
(3-{[5-(4-Hexyl-phenyl)-6-phenyl-pyridin-2-ylmethyl]-amino}-propyl)-phosphonic acid;
[1-(4-Hexyl-[1,1′;2′,1″]terphenyl-4′-ylmethyl)-pyrrolidin-3-yl]-phosphonic acid monoethyl ester;
[1-(4-Hexyl-[1,1′;2′,1″]terphenyl-4′-ylmethyl)-pyrrolidin-3-yl]-phosphonic acid;
(3-{[6-(6-Octyl-pyridin-3-yl)-biphenyl-3-ylmethyl]-amino}-propyl)-phosphonic acid;
(3-{[5-(4-Pentyloxy-phenyl)-6-phenyl-pyridin-2-ylmethyl]-amino}-propyl)-phosphonic acid;
(3-{[6-(4-Fluoro-phenyl)-5-(4-pentyloxy-phenyl)-pyridin-2-ylmethyl]-amino}-propyl)-phosphonic acid;
(3-{[6-(3-Fluoro-phenyl)-5-(4-pentyloxy-phenyl)-pyridin-2-ylmethyl]-amino}-propyl)-phosphonic acid;
(3-{[4-(3-Phenyl-propyl)-[1,1′;2′,1″]terphenyl-4′-ylmethyl]-amino}-propyl)-phosphonic acid;
(3-{[4-(3,3,4,4,5,5,6,6,6-Nonafluoro-hexyl)-[1,1′;2′,1″]terphenyl-4′-ylmethyl]-amino}-propyl)-phosphonic acid;
(3-{[5-(4-Hexyl-3-propyl-phenyl)-6-phenyl-pyridin-2-ylmethyl]-amino}-propyl)-phosphonic acid;
(3-{[6-(3-Chloro-phenyl)-5-(4-hexyl-3-propyl-phenyl)-pyridin-2-ylmethyl]-amino}-propyl)-phosphonic acid;
[3-({6-Phenyl-5-[4-(3-phenyl-propyl)-phenyl]-pyridin-2-ylmethyl}-amino)-propyl]-phosphonic acid;
[3-({6-(3-Chloro-phenyl)-5-[4-(3-phenyl-propyl)-phenyl]-pyridin-2-ylmethyl}-amino)-propyl]-phosphonic acid;
(3-{[5-(4-Hexyl-phenyl)-6-(3-hydroxy-phenyl)-pyridin-2-ylmethyl]-amino}-propyl)-phosphonic acid;
(3-{[5-(4-Hexyl-phenyl)-6-thiophen-2-yl-pyridin-2-ylmethyl]-amino}-propyl)-phosphonic acid;
(3-{[5-(4-Hexyl-phenyl)-6-thiophen-3-yl-pyridin-2-ylmethyl]-amino}-propyl)-phosphonic acid;
(3-{[6-Furan-2-yl-5-(4-hexyl-phenyl)-pyridin-2-ylmethyl]-amino}-propyl)-phosphonic acid;
(3-{[5-(4-Hexyl-phenyl)-6-oxazol-4-yl-pyridin-2-ylmethyl]-amino}-propyl)-phosphonic acid;
(3-{[5-(4-Hexyl-phenyl)-6-thiazol-2-yl-pyridin-2-ylmethyl]-amino}-propyl)-phosphonic acid;
[3-({5-[4-(3-Phenyl-propyl)-phenyl]-6-thiophen-2-yl-pyridin-2-ylmethyl}-amino)-propyl]-phosphonic acid;
(3-{[5-(4-Hexyl-3-propyl-phenyl)-6-thiophen-2-yl-pyridin-2-ylmethyl]-amino}-propyl)-phosphonic acid;
(3-{[3-(4-Hexyl-phenyl)-[2,3′]bipyridinyl-6-ylmethyl]-amino}-propyl)-phosphonic acid;
(3-{[6-(5-Fluoro-thiophen-2-yl)-5-(4-hexyl-phenyl)-pyridin-2-ylmethyl]-amino}-propyl)-phosphonic acid.

Preferred compounds of the invention are:
(3-{[5-(4-Hexyl-3-propyl-phenyl)-6-phenyl-pyridin-2-ylmethyl]-amino}-propyl)-phosphonic acid;
[3-({6-Phenyl-5-[4-(3-phenyl-propyl)-phenyl]-pyridin-2-ylmethyl}-amino)-propyl]-phosphonic acid;
(3-{[5-(4-Hexyl-phenyl)-6-thiophen-2-yl-pyridin-2-ylmethyl]-amino}-propyl)-phosphonic acid;
(3-{[5-(4-Hexyl-phenyl)-6-thiophen-3-yl-pyridin-2-ylmethyl]-amino}-propyl)-phosphonic acid;
(3-{[6-Furan-2-yl-5-(4-hexyl-phenyl)-pyridin-2-ylmethyl]-amino}-propyl)-phosphonic acid;
(3-{[5-(4-Hexyl-phenyl)-6-oxazol-4-yl-pyridin-2-ylmethyl]-amino}-propyl)-phosphonic acid;
(3-{[5-(4-Hexyl-phenyl)-6-thiazol-2-yl-pyridin-2-ylmethyl]-amino}-propyl)-phosphonic acid;
[3-({5-[4-(3-Phenyl-propyl)-phenyl]-6-thiophen-2-yl-pyridin-2-ylmethyl}-amino)-propyl]-phosphonic acid;
(3-{[5-(4-Hexyl-3-propyl-phenyl)-6-thiophen-2-yl-pyridin-2-ylmethyl]-amino}-propyl)-phosphonic acid;
(3-{[6-(5-Fluoro-thiophen-2-yl)-5-(4-hexyl-phenyl)-pyridin-2-ylmethyl]-amino}-propyl)-phosphonic acid.

Some compounds of Formula I and some of their intermediates have at least one stereogenic center in their structure. This stereogenic center may be present in an R or S configuration, said R and S notation is used in correspondence with the rules described in Pure Appli. Chem. (1976), 45, 11-13.

The term “pharmaceutically acceptable salts” refers to salts or complexes that retain the desired biological activity of the above identified compounds and exhibit minimal or no undesired toxicological effects. The “pharmaceutically acceptable salts” according to the invention include therapeutically active, non-toxic base or acid salt forms, which the compounds of Formula I are able to form.

The acid addition salt form of a compound of Formula I that occurs in its free form as a base can be obtained by treating the free base with an appropriate acid such as an inorganic, for example, a hydrohalic such as hydrochloric acid, hydrobromic acid, sulfuric acid, phosphoric acid, nitric acid and the like; or an organic acid such as for example, acetic, hydroxyacetic, propanoic, lactic, pyruvic, malonic, fumaric acid, maleic acid, oxalic acid, tartaric acid, succinic acid, malic acid, ascorbic acid, benzoic acid, tannic acid, pamoic acid, citric, methylsulfonic, ethanesulfonic, benzenesulfonic, formic and the like (Handbook of Pharmaceutical Salts, P. Heinrich Stahal & Camille G. Wermuth (Eds), Verlag Helvetica Chemica Acta-Zürich, 2002, 329-345).

Compounds of Formula I and their salts can be in the form of a solvate, which is included within the scope of the present invention. Such solvates include for example hydrates, alcoholates and the like.

With respect to the present invention reference to a compound or compounds, is intended to encompass that compound in each of its possible isomeric forms and mixtures thereof unless the particular isomeric form is referred to specifically.

Compounds according to the present invention may exist in different polymorphic forms. Although not explicitly indicated in the above formula, such forms are intended to be included within the scope of the present invention.

The compounds of the invention are indicated for use in treating or preventing conditions in which there is likely to be a component involving the sphingosine-1-phosphate receptors.

In another embodiment, there are provided pharmaceutical compositions including at least one compound of the invention in a pharmaceutically acceptable carrier.

In a further embodiment of the invention, there are provided methods for treating disorders associated with modulation of sphingosine-1-phosphate receptors. Such methods can be performed, for example, by administering to a subject in need thereof a pharmaceutical composition containing a therapeutically effective amount of at least one compound of the invention.

These compounds are useful for the treatment of mammals, including humans, with a range of conditions and diseases that are alleviated by S1P modulation: not limited to the treatment of diabetic retinopathy, other retinal degenerative conditions, dry eye, angiogenesis and wounds.

Therapeutic utilities of S1P modulators are ocular diseases, such as but not limited to: wet and dry age-related macular degeneration, diabetic retinopathy, retinopathy of prematurity, retinal edema, geographic atrophy, glaucomatous optic neuropathy, chorioretinopathy, hypertensive retinopathy, ocular ischemic syndrome, prevention of inflammation-induced fibrosis in the back of the eye, various ocular inflammatory diseases including uveitis, scleritis, keratitis, and retinal vasculitis; or systemic vascular barrier related diseases such as but not limited to: various inflammatory diseases, including acute lung injury, its prevention, sepsis, tumor metastasis, atherosclerosis, pulmonary edemas, and ventilation-induced lung injury; or autoimmune diseases and immunosuppression such as but not limited to: rheumatoid arthritis, Crohn's disease, Graves' disease, inflammatory bowel disease, multiple sclerosis, Myasthenia gravis, Psoriasis, ulcerative colitis, antoimmune uveitis, renal ischemia/perfusion injury, contact hypersensitivity, atopic dermititis, and organ transplantation; or allergies and other inflammatory diseases such as but not limited to: urticaria, bronchial asthma, and other airway inflammations including pulmonary emphysema and chronic obstructive pulmonary diseases; or cardiac protection such as but not limited to: ischemia reperfusion injury and atherosclerosis; or wound healing such as but not limited to: scar-free healing of wounds from cosmetic skin surgery, ocular surgery, GI surgery, general surgery, oral injuries, various mechanical, heat and burn injuries, prevention and treatment of photoaging and skin ageing, and prevention of radiation-induced injuries; or bone formation such as but not limited to: treatment of osteoporosis and various bone fractures including hip and ankles; or anti-nociceptive activity such as but not limited to: visceral pain, pain associated with diabetic neuropathy, rheumatoid arthritis, chronic knee and joint pain, tendonitis, osteoarthritis, neuropathic pains; or central nervous system neuronal activity in Alzheimer's disease, age-related neuronal injuries; or in organ transplant such as renal, corneal, cardiac or adipose tissue transplant.

In still another embodiment of the invention, there are provided methods for treating disorders associated with modulation of sphingosine-1-phosphate receptors. Such methods can be performed, for example, by administering to a subject in need thereof a therapeutically effective amount of at least one compound of the invention, or any combination thereof, or pharmaceutically acceptable salts, hydrates, solvates, crystal forms and individual isomers, enantiomers, and diastereomers thereof.

The present invention concerns the use of a compound of Formula I or a pharmaceutically acceptable salt thereof, for the manufacture of a medicament for the treatment of ocular disease, wet and dry age-related macular degeneration, diabetic retinopathy, retinopathy of prematurity, retinal edema, geographic atrophy, glaucomatous optic neuropathy, chorioretinopathy, hypertensive retinopathy, ocular ischemic syndrome, prevention of inflammation-induced fibrosis in the back of the eye, various ocular inflammatory diseases including uveitis, scleritis, keratitis, and retinal vasculitis; or systemic vascular barrier related diseases, various inflammatory diseases, including acute lung injury, its prevention, sepsis, tumor metastasis, atherosclerosis, pulmonary edemas, and ventilation-induced lung injury; or autoimmune diseases and immunosuppression, rheumatoid arthritis, Crohn's disease, Graves' disease, inflammatory bowel disease, multiple sclerosis, Myasthenia gravis, Psoriasis, ulcerative colitis, antoimmune uveitis, renal ischemia/perfusion injury, contact hypersensitivity, atopic dermititis, and organ transplantation; or allergies and other inflammatory diseases, urticaria, bronchial asthma, and other airway inflammations including pulmonary emphysema and chronic obstructive pulmonary diseases; or cardiac protection, ischemia reperfusion injury and atherosclerosis; or wound healing, scar-free healing of wounds from cosmetic skin surgery, ocular surgery, GI surgery, general surgery, oral injuries, various mechanical, heat and burn injuries, prevention and treatment of photoaging and skin ageing, and prevention of radiation-induced injuries; or bone formation, treatment of osteoporosis and various bone fractures including hip and ankles; or anti-nociceptive activity, visceral pain, pain associated with diabetic neuropathy, rheumatoid arthritis, chronic knee and joint pain, tendonitis, osteoarthritis, neuropathic pains; or central nervous system neuronal activity in Alzheimer's disease, age-related neuronal injuries; or in organ transplant such as renal, corneal, cardiac or adipose tissue transplant.

The actual amount of the compound to be administered in any given case will be determined by a physician taking into account the relevant circumstances, such as the severity of the condition, the age and weight of the patient, the patient's general physical condition, the cause of the condition, and the route of administration.

The patient will be administered the compound orally in any acceptable form, such as a tablet, liquid, capsule, powder and the like, or other routes may be desirable or necessary, particularly if the patient suffers from nausea. Such other routes may include, without exception, transdermal, parenteral, subcutaneous, intranasal, via an implant stent, intrathecal, intravitreal, topical to the eye, back to the eye, intramuscular, intravenous, and intrarectal modes of delivery. Additionally, the formulations may be designed to delay release of the active compound over a given period of time, or to carefully control the amount of drug released at a given time during the course of therapy.

In another embodiment of the invention, there are provided pharmaceutical compositions including at least one compound of the invention in a pharmaceutically acceptable carrier therefor. The phrase “pharmaceutically acceptable” means the carrier, diluent or excipient must be compatible with the other ingredients of the formulation and not deleterious to the recipient thereof.

Pharmaceutical compositions of the present invention can be used in the form of a solid, a solution, an emulsion, a dispersion, a micelle, a liposome, and the like, wherein the resulting composition contains one or more compounds of the present invention, as an active ingredient, in admixture with an organic or inorganic carrier or excipient suitable for enteral or parenteral applications. Invention compounds may be combined, for example, with the usual non-toxic, pharmaceutically acceptable carriers for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used. Invention compounds are included in the pharmaceutical composition in an amount sufficient to produce the desired effect upon the process or disease condition.

Pharmaceutical compositions containing invention compounds may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of a sweetening agent such as sucrose, lactose, or saccharin, flavoring agents such as peppermint, oil of wintergreen or cherry, coloring agents and preserving agents in order to provide pharmaceutically elegant and palatable preparations. Tablets containing invention compounds in admixture with non-toxic pharmaceutically acceptable excipients may also be manufactured by known methods. The excipients used may be, for example, (1) inert diluents such as calcium carbonate, lactose, calcium phosphate or sodium phosphate; (2) granulating and disintegrating agents such as corn starch, potato starch or alginic acid; (3) binding agents such as gum tragacanth, corn starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

In some cases, formulations for oral use may be in the form of hard gelatin capsules wherein the invention compounds are mixed with an inert solid diluent, for example, calcium carbonate, calcium phosphate or kaolin. They may also be in the form of soft gelatin capsules wherein the invention compounds are mixed with water or an oil medium, for example, peanut oil, liquid paraffin, or olive oil.

The pharmaceutical compositions may be in the form of a sterile injectable suspension. This suspension may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Sterile, fixed oils are conventionally employed as a solvent or suspending medium. For this purpose any bland fixed oil may be employed including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate or the like. Buffers, preservatives, antioxidants, and the like can be incorporated as required.

Invention compounds may also be administered in the form of suppositories for rectal administration of the drug. These compositions may be prepared by mixing the invention compounds with a suitable non-irritating excipient, such as cocoa butter, synthetic glyceride esters of polyethylene glycols, which are solid at ordinary temperatures, but liquify and/or dissolve in the rectal cavity to release the drug.

Since individual subjects may present a wide variation in severity of symptoms and each drug has its unique therapeutic characteristics, the precise mode of administration and dosage employed for each subject is left to the discretion of the practitioner.

The compounds and pharmaceutical compositions described herein are useful as medicaments in mammals, including humans, for treatment of diseases and or alleviations of conditions which are responsive to treatment by agonists or functional antagonists of sphingosine-1-phosphate receptors. Thus, in further embodiments of the invention, there are provided methods for treating a disorder associated with modulation of sphingosine-1-phosphate receptors. Such methods can be performed, for example, by administering to a subject in need thereof a pharmaceutical composition containing a therapeutically effective amount of at least one invention compound. As used herein, the term “therapeutically effective amount” means the amount of the pharmaceutical composition that will elicit the biological or medical response of a subject in need thereof that is being sought by the researcher, veterinarian, medical doctor or other clinician. In some embodiments, the subject in need thereof is a mammal. In some embodiments, the mammal is human.

The present invention concerns also processes for preparing the compounds of Formula I. The compounds of formula I according to the invention can be prepared analogously to conventional methods as understood by the person skilled in the art of synthetic organic chemistry. The synthetic scheme set forth below, illustrates how compounds according to the invention can be made. Those skilled in the art will be able to routinely modify and/or adapt the following scheme to synthesize any compounds of the invention covered by Formula I.

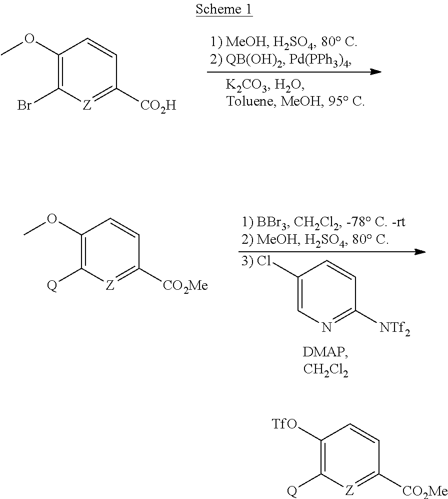

In Scheme 1, the commercially available carboxylic acid was esterified followed by a Suzuki coupling with available aryl boronic acids to give rise to the biaryl methoxy ester. Demethylation and re-esterification resulted in the corresponding phenolic ester, which was converted to a triflate.

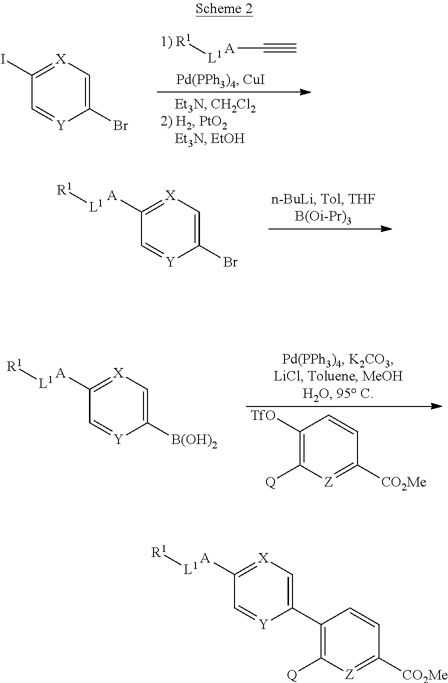

In Scheme 2, Sonogashira coupling followed by reduction of the resulting alkyne afforded the substituted aryl halide. Conversion to the boronic acid followed Suzuki coupling with the resulting aryl triflate from Scheme 1 afforded the desired triaryl ester.

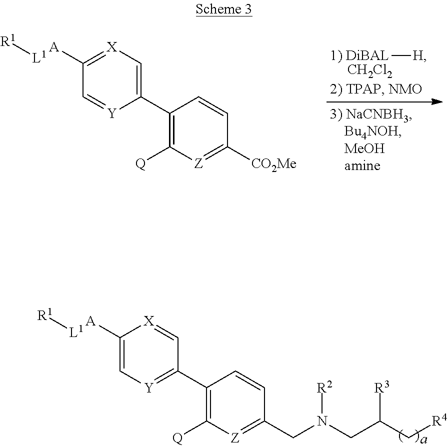

In Scheme 3, the desired final compound of Formula I, was afforded in three final steps from the triaryl ester. Reduction of the ester to the alcohol and subsequent oxidation afforded the corresponding aldehyde. Reductive amination of this aldehyde yielded the final product.

### DETAILED DESCRIPTION OF THE INVENTION

It is to be understood that both the foregoing general description and the following detailed description are exemplary and explanatory only and are not restrictive of the invention claimed. As used herein, the use of the singular includes the plural unless specifically stated otherwise.

It will be readily apparent to those skilled in the art that some of the compounds of the invention may contain one or more asymmetric centers, such that the compounds may exist in enantiomeric as well as in diastereomeric forms. Unless it is specifically noted otherwise, the scope of the present invention includes all enantiomers, diastereomers and racemic mixtures. Some of the compounds of the invention may form salts with pharmaceutically acceptable acids or bases, and such pharmaceutically acceptable salts of the compounds described herein are also within the scope of the invention.

The present invention includes all pharmaceutically acceptable isotopically enriched compounds. Any compound of the invention may contain one or more isotopic atoms enriched or different than the natural ratio such as deuterium ²H (or D) in place of hydrogen ¹H (or H) or use of ¹³C enriched material in place of ¹²C and the like. Similar substitutions can be employed for N, O and S. The use of isotopes may assist in analytical as well as therapeutic aspects of the invention. For example, use of deuterium may increase the in vivo half-life by altering the metabolism (rate) of the compounds of the invention. These compounds can be prepared in accord with the preparations described by use of isotopically enriched reagents.

The following examples are for illustrative purposes only and are not intended, nor should they be construed as limiting the invention in any manner. Those skilled in the art will appreciate that variations and modifications of the following examples can be made without exceeding the spirit or scope of the invention.

As will be evident to those skilled in the art, individual isomeric forms can be obtained by separation of mixtures thereof in conventional manner. For example, in the case of diasteroisomeric isomers, chromatographic separation may be employed.

The IUPAC names of the compounds mentioned in the examples were generated with ACD version 8.

Unless specified otherwise in the examples, characterization of the compounds is performed according to the following methods:

NMR spectra are recorded on 300 or 600 MHz Varian and acquired at room temperature. Chemical shifts are given in ppm referenced either to internal trimethylsilyl or to the residual solvent signal.

All the reagents, solvents, catalysts for which the synthesis is not described are purchased from chemical vendors such as Sigma Aldrich, Fluka, Bio-Blocks, Ryan Scientific, Syn Chem, Chem-Impex, Aces Pharma, however some known intermediates, for which the CAS registry number [CAS #] are mentioned, were prepared in-house following known procedures.

Usually the compounds of the invention were purified by flash column chromatography using a gradient solvent system of methanol/dichloromethane unless otherwise reported.

The following abbreviations are used in the examples:

DMF N,N-dimethylformamide

NaOH sodium hydroxide

CD₃OD deuterated methanol

HCl hydrochloric acid

CDCl₃ deuterated chloroform

DMSO-d₆ deuterated dimethyl sulfoxide

CDl 1,1′-carbonyldiimidazole

Et₂Zn diethylzinc

NH₄Cl ammonium chloride

CH₂Cl₂ dichloromethane

K₂CO₃ potassium carbonate

MPLC medium pressure liquid chromatography

THF tetrahydrofuran

[IrCl(cod)]₂ di-μ-chlorobis(1,5-cyclooctadiene)diiridium(I)

ClCH₂I chloroiodomethane

RT room temperature

MeOH methanol

DMAP 4-Dimethylaminopyridine

MgSO₄ magnesium sulfate

LiCl lithium chloride

DIBAL-H Diisobutylaluminium hydride

NMO N-Methylmorpholine-N-Oxide

LDA Lithium diisopropylamide

MTBE Methyl tert-butyl ether

Na₂SO₄ sodium sulfate

dppp 1,3-Bis(diphenylphosphino)propane

Those skilled in the art will be able to routinely modify and/or adapt the following schemes to synthesize any compound of the invention covered by Formula I.

Some compounds of this invention can generally be prepared in one step from commercially available literature starting materials.

### Example 1

### Intermediate 1

### Methyl 6-methoxybiphenyl-3-carboxylate

To a solution of methyl 3-bromo-4-methoxybenzoate (13 g, 53 mmol) in toluene (500 mL), methanol (65 mL), and water (106 mL) was added phenyl boronic acid (7.8 g, 63.6 mmol) and potassium carbonate (14.6 g, 106 mmol) and bubbled with argon for 6 min. Tetrakis(triphenylphosphine)palladium(0) (370 mg) was added and bubbled with argon for another 2 min. The reaction mixture was then heated to 95° C. for 20 h with stirring. After cooling to RT, the two phases were separated and the aqueous layer was extracted with ether, dried with magnesium sulfate, and concentrated. Purification by MPLC (5% ethyl acetate in hexanes) gave 12.2 g of the desired product as an off white solid.

### Example 2

### Intermediate 2

### Methyl 6-{[(trifluoromethyl)sulfonyl]oxy}biphenyl-3-carboxylate

To a solution of Intermediate 1 (12.2 g, 50.4 mmol) in dichloromethane (200 mL) at −78° C. was added boron tribromide (100 mL, 1M in dichloromethane) dropwise with stirring. The reaction mixture was warmed to RT and stirred for 16 h, after which time, the reaction mixture was cooled to −78° C. and boron tribromide (20 mL, 1M in dichloromethane) was added and stirred at RT for another 6 h. Cooling to −10° C., the reaction mixture was quenched with a saturated solution of sodium bicarbonate. The layers were separated and the aqueous layer was acidified with 1N HCl. Extraction of the aqueous layer with ethyl acetate followed by combination of the organic layers, washed with brine, dried with magnesium sulfate, and concentrated to afford 6 g corresponding phenolic acid as colorless foam.

A solution of the resulting carboxylic acid (6 g, 26.3 mmol) in MeOH (80 mL) was added and fuming sulfuric acid (3 mL) dropwise. After heating to 80° C. for 16 h, the reaction mixture was cooled to RT and concentrated under reduced pressure. The residue was diluted with water and extracted with ethyl acetate, dried over magnesium sulfate and concentrated under reduced pressure to give 5.17 g desired phenolic ester.

To a solution of the resulting phenolic ester (5.17 g, 22.6 mmol) in dichloromethane (500 mL) was added N-(5-chloro-2-pyridyl)bis(trifluoromethanesulfonimide) (13.3 g, 34 mmol) and DMAP (5.5 g, 45.2 mmol) with stirring. After 16 h at RT, the reaction mixture was quenched with water. The aqueous layer was extracted with ethyl acetate, dried (MgSO₄), and concentrated under reduced pressure. Purification by MPLC (5% ethyl acetate in hexanes) gave rise to 6.67 g of the title compound as a colorless oil.

¹H NMR (300 MHz, CDCl₃) δ 8.19 (d, J=2.05 Hz, 1H), 8.11 (dd, J=2.20, 8.64 Hz, 1H), 7.46-7.50 (m, 6H), 3.96 (s, 3H).

### Example 3

### Intermediate 3

### Methyl 4-hexyl-1,1′:2′,1″-terphenyl-4′-carboxylate

To a solution of aryl bromide (2.8 g, 11.6 mmol) in THF (100 mL) at −78° C. was added t-butyllithium (1.7 M in pentane, 13.8 mL) slowly dropwise. After stirring at −78° C. for 1 h, trimethyl borate (2.63 mL, 23.56 mmol) was added. The reaction mixture was warmed to RT over 2 h. After stirring at RT for 15 min, the reaction mixture was quenched with saturated solution of ammonium chloride and extracted with ethyl acetate. The combined organic layers were washed with HCl (10% solution), brine, and dried (MgSO₄), filtered, and concentrated under reduce pressure to give 2.22 g boronic acid. A solution of the resulting boronic acid (2.22 g) and Intermediate 2 (3.8 g, 10.7 mmol) in toluene (200 mL) were added potassium carbonate (2.95 g, 21.4 mmol) and LiCl (454 mg) with stirring. After bubbling with Ar for 10 min, tetrakis(triphenylphosphine) palladium(0) (370 mg) was added and heated to 95° C. for 16 h. After the reaction mixture was cooled to RT, it was diluted with water and extracted with ethyl acetate. The combined organic layers were washed with brine, and dried (MgSO₄), filtered, and concentrated under reduce pressure. The residue was purified by MPLC (0-10% ethyl acetate in hexanes) gave 2.32 g of ester as a colorless oil.

¹H NMR (300 MHz, CDCl₃) δ 8.09 (d, J=1.76 Hz, 1H), 8.05 (dd, J=1.76, 7.91 Hz, 1H), 7.50 (d, J=7.91 Hz, 1H), 7.20-7.24 (m, 3H), 7.13-7.17 (m, 2H), 7.04 (s, 4H), 3.94 (s, 3H), 2.56 (t, J=7.62 Hz, 2H), 1.53-1.63 (m, 2H), 1.25-1.33 (m, 6H), 0.88 (t, J=6.45 Hz, 3H)

Intermediates 4-8 were prepared from Intermediate 2 and the corresponding aryl bromide derivatives, in a similar manner to the method described in Example 3 for Intermediate 3. The results are described below in Table 1.

TABLE 1

| Interm. | | |
|---|---|---|
| number | IUPAC name | ¹H NMR δ (ppm) for Intermediate |
| 4 | Methyl-4-octyl-1,1′:2′,1″- | ¹H NMR (300 MHz, CDCl₃) δ 8.10 (d, J = 1.76 Hz, |
| | terphenyl-4′-carboxylate | 1H), 8.05 (dd, J = 1.76, 7.91 Hz, 1H), |
| | | 7.49 (d, J = 7.91 Hz, 1H), 7.20-7.25 (m, 3H), |
| | | 7.12-7.17 (m, 3H), 7.03 (s, 4H), 3.94 (s, 3H), |
| | | 2.52-2.58 (m, 2H), 1.53-1.62 (m, 2H), 1.28 (d, J = 5.57 Hz, |
| | | 10H), 0.88 (t, J = 6.45 Hz, 3H) |
| 5 | Methyl-6-(6-hexylpyridin- | ¹H NMR (300 MHz, CDCl₃) δ 8.34-8.40 (m, J = 0.59, |
| | 3-yl)biphenyl-3- | 2.34 Hz, 1H), 8.12 (d, J = 1.47 Hz, 1H), |
| | carboxylate | 8.09 (dd, J = 1.76, 7.91 Hz, 1H), 7.49 (d, J = 7.91 Hz, |
| | | 2H), 7.11-7.29 (m, 5H), 6.97 (d, J = 8.20 Hz, |
| | | 1H), 3.95 (s, 3H), 2.75 (t, J = 7.62 Hz, |
| | | 2H), 1.64-1.74 (m, 2H), 1.26-1.36 (m, 6H), |
| | | 0.87 (t, J = 6.74 Hz, 3H) |
| 6 | Methyl-6-(6-octylpyridin- | ¹H NMR (300 MHz, CDCl₃) δ 8.37 (dd, J = 0.88, |
| | 3-yl)biphenyl-3- | 2.34 Hz, 3H), 8.08-8.13 (m, 2H), 7.49 (d, J = 7.91 Hz, |
| | carboxylate | 1H), 7.22-7.29 (m, 4H), |
| | | 7.12-7.15 (m, 2H), 6.97 (dd, J = 0.59, 7.91 Hz, 1H), |
| | | 3.95 (s, 3H), 2.74 (dd, J = 7.60 Hz, 2H), |
| | | 1.64-1.74 (m, 2H), 1.23-1.35 (m, 10H), 0.88 (t, J = 6.45 Hz, |
| | | 3H) |
| 7 | Methyl-4- | ¹H NMR (300 MHz, CDCl₃) δ 8.04-8.11 (m, |
| | (3,3,4,4,5,5,6,6,6- | 2H), 7.48 (d, J = 7.91 Hz, 2H), 7.21-7.25 (m, |
| | nonafluorohexyl)- | 3H), 7.11-7.17 (m, 2H), 7.09 (s, 3H), 3.94 (s, |
| | 1,1′:2′,1″-terphenyl-4′- | 3H), 2.85-2.91 (m, 2H), 2.26-2.44 (m, 2H) |
| | carboxylate | |
| 8 | Methyl-4-(3- | ¹H NMR (300 MHz, CDCl₃) δ 8.10 (d, J = 1.47 Hz, |
| | phenylpropyl)-1,1′:2′,1″- | 1H), 8.05 (dd, J = 1.76, 7.91 Hz, 1H), |
| | terphenyl-4′-carboxylate | 7.46-7.51 (m, 2H), 7.13-7.31 (m, 9H), 7.04 (s, 4H), |
| | | 3.94 (s, 3H), 2.61 (t, J = 7.62 Hz, 4H), |
| | | 1.88-1.98 (m, 2H) |

### Example 4

### Intermediate 9

### Ethyl 6-(2-furyl)-5-(4-hexylphenyl)pyridine-2-carboxylate

To a solution of methyl 3-propyl-4-{[(trifluoromethyl)sulfonyl]oxy}benzoate (2.09 g, 7.11 mmol) and 1-hexyne (1.12 mL) in DMF (17.5 mL) and triethyl amine (3.5 mL) was added dppp (100 mg, 0.14 mmol). After heating to 95° C. with stirring for 16 h, the reaction mixture was cooled to RT, diluted with diethyl ether and washed with water. The ethereal layer was washed with brine, and dried (MgSO₄), filtered, and concentrated under reduce pressure. The residue was purified by MPLC (3% ethyl acetate in hexanes) to give 11.9 g ethyl 6-(2-furyl)-5-(4-hexylphenyl)pyridine-2-carboxylate as a brown oil.

¹H NMR (300 MHz, CDCl₃) δ 7.84 (d, J=1.76 Hz, 1H), 7.77 (dd, J=1.61, 8.06 Hz, 1H), 7.41 (d, J=7.91 Hz, 1H), 3.90 (s, 3H), 2.77 (t, J=7.91 Hz, 2H), 2.47 (t, J=6.70 Hz, 2H), 1.44-1.74 (m, 6H), 0.96 (t, J=7.33 Hz, 6H).

### Example 5

### Intermediate 10

### Methyl 4-hexyl-3-propylbenzoate

To a solution of Intermediate 9 (2.5 g, 9.7 mmol) in ethanol (110 mL) was added palladium hydroxide on carbon (20% wt on carbon, 700 mg). After stirring at RT under hydrogen balloon atmosphere for 16 h, the reaction mixture was filtered through celite and concentrated under reduced pressure. Filtration through a short plug of silica gel afforded 4.7 g of methyl 4-hexyl-3-propylbenzoate as a brown oil.

¹H NMR (300 MHz, CDCl₃) δ 7.82 (d, J=1.47 Hz, 1H), 7.78 (dd, J=1.76, 7.91 Hz, 1H), 7.20 (d, J=7.91 Hz, 1H), 3.89 (s, 3H), 2.59-2.67 (m, 4H), 1.52-1.69 (m, 4H), 1.22-1.41 (m, 6H), 0.99 (t, J=7.33 Hz, 3H), 0.89 (t, J=6.45 Hz, 3H)

### Example 6

### Intermediate 11

### (4-hexyl-3-propylphenyl)methanol

To a solution of Intermediate 10 6.4 g, 24.4 mmol) in dichloromethane (230 mL) at −78° C. was added DIBAL-H (1.0 M in dichloromethane, 58.6 mL, 58.6 mmol). The reaction was warmed to RT over for 20 h with stirring and was quenched at −10° C. with methanol and 10% solution of HCl. The mixture was diluted with water and the aqueous layer was extracted with ethyl acetate. The combined organic layers were washed with brine, dried over MgSO₄, and concentrated under reduced pressure to afford 5.4 g of (4-hexyl-3-propylphenyl)methanol as a yellow oil.

¹H NMR (300 MHz, CDCl₃) δ 7.11-7.18 (m, 3H), 4.63 (s, 2H), 2.59 (td, J=1.32, 7.84 Hz, 4H), 1.50-1.68 (m, 4H), 1.22-1.40 (m, 6H), 0.99 (t, J=7.33 Hz, 3H), 0.89 (t, J=6.74 Hz, 3H)

### Example 7

### Intermediate 12

### 4-hexyl-3-propylbenzaldehyde

To a solution of Intermediate 11 (4.1 g, 17.5 mmol), NMO (5.1 g, 43 mmol), and 4 A molecular sieves (4 g) in dichloromethane (170 mL) and acetonitrile (22 mL) was added tetrapropylammonium perruthenate (TPAP, 320 mg). After stirring at RT for 2 h, the reaction mixture was filtered through a short column of silica gel, eluted with ethyl acetate and concentrated under reduced pressure. Purification by MPLC (0-20% ethyl acetate in hexanes) gave rise to 2.96 g 4-hexyl-3-propylbenzaldehyde as a yellow oil.

¹H NMR (300 MHz, CDCl₃) δ 9.95 (s, 1H), 7.66 (d, J=1.47 Hz, 1H), 7.63 (dd, J=1.80, 7.62 Hz, 1H), 7.30 (d, J=7.62 Hz, 1H), 2.63-2.70 (m, 4H), 1.54-1.71 (m, 4H), 1.26-1.43 (m, 6H), 1.00 (t, J=7.33 Hz, 3H), 0.90 (t, J=6.70 Hz, 3H)

### Example 8

### Intermediate 13

### (2E)-3-(4-hexyl-3-propylphenyl)acrylaldehyde

To a solution of LDA (1.5M in cyclohexane, 9 mL, 13.5 mmol) in THF (28 mL) at 0° C., was added a solution of 2-methyl-N-[2-(triethylsilyl)ethylidene]-2-propanamine (2.9 g, 13.5 mmol) in THF (6 mL) dropwise and stirred for 30 min. The reaction mixture was cooled to −78° C. and a solution of Intermediate 12 (2.6 g, 12.3 mmol) in THF (6 mL) was added dropwise. After warming to RT over 3.5 h, the reaction mixture was quenched with citric acid (20% solution, 40 mL) and stirred for another 16 h. The mixture was washed with brine, extracted with diethyl ether, dried over MgSO₄, and concentrated under reduced pressure. Purification of the crude product by MPLC (10% ethyl acetate in hexanes) afforded 3.8 g (2E)-3-(4-hexyl-3-propylphenyl)acrylaldehyde

¹H NMR (300 MHz, CDCl₃) δ 9.68 (d, J=7.91 Hz, 1H), 7.44 (d, J=16.12 Hz, 1H), 7.31-7.36 (m, 2H), 7.20 (d, J=8.50 Hz, 1H), 6.69 (dd, J=7.62, 15.82 Hz, 1H), 2.59-2.66 (m, 4H), 1.52-1.69 (m, 4H), 1.28-1.43 (m, 6H), 1.00 (t, J=7.33 Hz, 3H), 0.90 (t, J=6.74 Hz, 3H)

### Example 9

### Intermediate 14

### (2E)-3-[4-(3-phenylpropyl)phenyl]acrylaldehyde

To a solution of 1-bromo-4-(3-phenylpropyl)-benzene (684 mg, 2.48 mmol) in DMF (10 mL) were added acrolein diethyl acetal (1.7 mL, 11.1 mmol), tetrabutylammonium acetate (1.87 g, 6.2 mmol), potassium carbonate (514 mg, 3.72 mmol), potassium chloride (185 mg, 2.48 mmol), and palladium(II) acetate (50 mg, 0.22 mmol). After stirring at 90° C. for 4 h, the reaction mixture was cooled to RT and HCl (2M, 15 mL) was added. After stirring for 10 min at RT, the mixture was extracted with MTBE and washed with water and brine, dried over MgSO₄ and concentrated under reduced pressure. Purification by MPLC (20% ethyl acetate in hexanes) gave 390 mg (2E)-3-[4-(3-phenylpropyl)phenyl]acrylaldehyde as a yellow oil.

¹H NMR (300 MHz, CDCl₃) δ 9.69 (d, J=7.62 Hz, 1H), 7.42-7.51 (m, 3H), 7.16-7.32 (m, 7H), 6.69 (dd, J=7.62, 15.82 Hz, 1H), 2.67 (q, J=8.20 Hz, 4H), 1.91-2.03 (m, 2H)

### Example 10

### Intermediate 15

### ethyl (2Z,4E)-2-azido-5-(4-hexyl-3-propylphenyl)penta-2,4-dienoate

To a freshly prepared solution of sodium ethoxide (76 mmol) at −10° C. was added a solution of ethyl azidoacetate (25% in ethanol, 39.2 mL, 76 mmol) followed by a solution of Intermediate 13 (3.25 g, 12.6 ml) in ethanol (45 mL). After stirring for 1 h at −10° C., the reaction mixture was quenched with water and brine and extracted with ethyl acetate. The combined organic layers were washed with water and brine, dried over MgSO4, and concentrated under reduced pressure. Purification by MPLC (10% ethyl acetate in hexanes) gave rise to 1.65 g ethyl (2Z,4E)-2-azido-5-(4-hexyl-3-propylphenyl)penta-2,4-dienoate as a yellow oil.

¹H NMR (300 MHz, CDCl₃) δ 7.23-7.26 (m, 2H), 7.11 (s, 2H), 6.73-6.81 (m, 2H), 4.33 (q, J=7.13 Hz, 2H), 2.59 (t, J=7.91 Hz, 2H), 2.59 (t, J=7.91 Hz, 2H), 1.51-1.68 (m, 4H), 1.37 (t, J=7.18 Hz, 3H), 1.24-1.43 (m, 6H), 1.00 (t, J=7.33 Hz, 3H), 0.90 (t, J=7.00 Hz, 3H).

Intermediates 16-18 were prepared from the corresponding starting materials, in a similar manner to the method described in Example 10 for Intermediate 15. The starting materials used and the results are described below in Table 2.

TABLE 2

| Interm. | | Starting | ¹H NMR δ (ppm) for |
|---|---|---|---|
| number | IUPAC name | Materials | Intermediate |
| 16 | ethyl (2Z,4E)-2-azido- | 2-Propenal, | ¹H NMR (300 MHz, CDCl₃) δ |
| | 5-(4- | 3-(4-hexyl | 7.40 (d, J = 7.91 Hz, 2H), 7.07-7.18 (m, |
| | hexylphenyl)penta- | phenyl)- | 3H), 6.80 (d, J = 15.53 Hz, 1H), |
| | 2,4-dienoate | CAS313690-31-2 | 6.75 (dd, J = 1.17, 11.14 Hz, 1H), |
| | | | 4.33 (q, J = 7.23 Hz, 2H), 2.60 (t, J = 7.60 Hz, |
| | | | 2H), 1.57-1.65 (m, 2H), |
| | | | 1.37 (t, J = 7.18 Hz, 3H), |
| | | | 1.24-1.38 (m, 6H), 0.88 (t, J = 7.03 Hz, |
| | | | 3H) |
| 17 | ethyl (2Z,4E)-2-azido- | Intermediate 14 | ¹H NMR (300 MHz, CDCl₃) δ |
| | 5-(4-(3- | | 7.40 (d, J = 8.21 Hz, 2H), 7.25-7.32 (m, |
| | phenylpropyl)phenyl)penta- | | 2H), 7.08-7.22 (m, 6H), |
| | 2,4-dienoate | | 6.73-6.82 (m, 2H), 4.33 (q, J = 7.13 Hz, |
| | | | 2H), 2.65 (t, J = 7.62 Hz, 4H), |
| | | | 1.90-2.01 (m, 2H), 1.37 (t, J = 7.18 Hz, |
| | | | 3H) |
| 18 | ethyl (2Z,4E)-2-azido- | 2-Propenal, | ¹H NMR (300 MHz, CDCl₃) δ |
| | 5-(4- | 3-[4-(pentyloxy)phenyl]- | 7.39-7.44 (m, 2H), 7.02 (dd, J = 11.70, |
| | pentyloxyphenyl)penta- | CAS 66049-89-6 | 14.94 Hz, 1H), 6.84-6.89 (m, 2H), |
| | 2,4-dienoate | | 6.73-6.79 (m, 2H), 4.32 (q, J = 7.13 Hz, |
| | | | 2H), 3.97 (t, J = 6.59 Hz, |
| | | | 2H), 1.74-1.84 (m, 2H), |
| | | | 1.36-1.48 (m, 4H), 1.38 (t, J = 7.20 Hz, |
| | | | 3H), 0.93 (t, J = 7.00 Hz, 3H) |

### Example 11

### Intermediate 19

### Ethyl(2Z,4E)-5-(4-hexyl-3-propylphenyl)-2-[(triphenylphosphoranylidene)amino]penta-2,4-dienoate

To a solution of Intermediate 15 (1.65 g, 4.47 mmol) in diethyl ether (22 mL) at 0° C. was added a solution of triphenylphosphine (1.17 g) in diethyl ether (11 mL). After stirring for 16 h at RT, the reaction mixture was concentrated under reduced pressure. Purification by MPLC (20% ethyl acetate in hexanes) gave 2.2 g ethyl (2Z,4E)-5-(4-hexyl-3-propylphenyl)-2-[(triphenylphosphoranylidene)amino]penta-2,4-dienoate as a yellow foam.

¹H NMR (300 MHz, CDCl₃) δ 7.64-7.80 (m, 7H), 7.39-7.51 (m, 9H), 7.23 (s, 1H), 7.13 (d, J=7.91 Hz, 1H), 7.05 (d, J=8.20 Hz, 1H), 6.73 (dd, J=3.81, 11.14 Hz, 1H), 6.60 (d, J=15.82 Hz, 1H), 3.89 (q, J=7.13 Hz, 2H), 2.57 (t, J=7.91 Hz, 4H), 1.51-1.70 (m, 4H), 1.26-1.43 (m, 6H), 0.96-1.06 (m, 6H), 0.85-0.92 (m, 3H).

Intermediates 20-22 were prepared from the corresponding starting materials, in a similar manner to the method described in Example 11 for Intermediate 19. The starting materials and the results are described below in Table 3.

TABLE 3

| Interm. | | Starting | ¹H NMR δ (ppm) for |
|---|---|---|---|
| number | IUPAC name | materials | Intermediate |
| 20 | ethyl (2Z,4E)-5-[4-(3- | Intermediate 17 | ¹H NMR (300 MHz, |
| | phenylpropyl)phenyl]-2- | | CDCl₃) δ 7.40 (d, J = 7.91 Hz, |
| | [(triphenylphosphoranylidene)amino]penta- | | 2H), 7.07-7.18 (m, |
| | 2,4- | | 3H), 6.80 (d, J = 15.53 Hz, |
| | dienoate | | 1H), 6.75 (dd, J = 1.17, |
| | | | 11.14 Hz, 1H), 4.33 (q, J = 7.23 Hz, |
| | | | 2H), 2.60 (t, J = 7.60 Hz, |
| | | | 2H), |
| | | | 1.57-1.65 (m, 2H), 1.37 (t, J = 7.18 Hz, |
| | | | 3H), 1.24-1.38 (m, |
| | | | 6H), 0.88 (t, J = 7.03 Hz, |
| | | | 3H) |
| 21 | ethyl (2Z,4E)-5-(4- | Intermediate 16 | ¹H NMR (300 MHz, |
| | hexylphenyl)-2- | | CDCl₃) δ 7.71-7.80 (m, |
| | [(triphenylphosphoranylidene)amino]penta- | | 6H), 7.66 (dd, J = 11.14, |
| | 2,4- | | 15.82 Hz, 1H), |
| | dienoate | | 7.39-7.53 (m, 9H), 7.29 (d, J = 8.21 Hz, |
| | | | 2H), 7.10 (s, 2H), |
| | | | 6.72 (dd, J = 3.66, 10.70 Hz, |
| | | | 1H), 6.62 (d, J = 15.82 Hz, |
| | | | 1H), 3.90 (q, J = 7.13 Hz, |
| | | | 2H), 2.57 (t, J = 7.60 Hz, |
| | | | 2H), 1.54-1.65 (m, 2H), |
| | | | 1.25-1.37 (m, 6H), |
| | | | 1.04 (t, J = 7.18 Hz, 3H), |
| | | | 0.88 (t, J = 7.00 Hz, 3H) |
| 22 | ethyl(2Z,4E)-5-(4-pentyloxyphenyl)- | Intermediate 18 | ¹H NMR (300 MHz, |
| | 2-[(triphenylphosphoranylidene)amino]penta- | | CDCl₃) δ 7.71-7.80 (m, |
| | 2,4-dienoate | | 6H), 7.39-7.60 (m, 10H), |
| | | | 7.29 (d, J = 8.50 Hz, 2H), |
| | | | 6.81 (d, J = 8.79 Hz, 2H), |
| | | | 6.72 (dd, J = 3.37, |
| | | | 10.99 Hz, 1H), 6.59 (d, J = 15.82 Hz, |
| | | | 1H), 3.96 (d, J = 13.19 Hz, |
| | | | 2H), 3.90 (q, J = 7.00 Hz, |
| | | | 2H), 1.73-1.83 (m, |
| | | | 2H), 1.32-1.50 (m, 4H), |
| | | | 1.04 (t, J = 7.18 Hz, 3H), |
| | | | 0.94 (t, J = 6.74 Hz, 3H) |

### Example 12

### Intermediate 23

### Ethyl 5-(4-hexylphenyl)-6-(3-thienyl)pyridine-2-carboxylate

To a solution of Intermediate 21 (766 mg, 1.36 mmol) in acetonitrile (20 mL) was added thiophene-3-carbaldehyde (0.12 mL, 1.36 mmol). After stirring at 65° C. for 16 h, the reaction mixture was concentrated under reduced pressure and purified by MPLC (10% ethyl acetate in hexanes) to afford 350 mg of ethyl 5-(4-hexylphenyl)-6-(3-thienyl)pyridine-2-carboxylate as a yellow oil.

¹H NMR (300 MHz, CDCl₃) δ 7.97 (d, J=7.91 Hz, 1H), 7.67 (d, J=7.91 Hz, 1H), 7.06-7.14 (m, 5H), 7.00-7.05 (m, 2H), 4.41 (q, J=7.03 Hz, 2H), 2.56 (t, J=7.91 Hz, 2H), 1.45-1.66 (m, 2H), 1.38 (t, J=7.03 Hz, 3H), 1.17-1.32 (m, 6H), 0.77-0.84 (m, 3H).

Intermediates 24-39 were prepared from the corresponding starting materials, in a similar manner to the method described in Example 12 for Intermediate 23. The starting materials and the results are described below in Table 4.

TABLE 4

| | | | ¹H NMR δ (ppm) for |
|---|---|---|---|
| Interm. number | IUPAC name | Starting material | Intermediate |
| 24 | Ethyl-5-(4-hexylphenyl)- | Intermediate 21 | ¹H NMR (300 MHz, CDCl₃) |
| | 6-phenylpyridine-2- | Benzaldehyde | δ 8.10 (d, J = 8.20 Hz, 1H), |
| | carboxylate | CAS 100-52-7 | 7.83 (d, J = 7.91 Hz, 1H), |
| | | | 7.38-7.42 (m, 2H), |
| | | | 7.20-7.26 (m, 2H), 7.08 (s, 5H), |
| | | | 4.49 (q, J = 7.23 Hz, 2H), |
| | | | 2.56-2.61 (m, 2H), |
| | | | 1.54-1.64 (m, 2H), 1.45 (t, J = 7.18 Hz, |
| | | | 3H), |
| | | | 1.25-1.35 (m, 6H), 0.88 (t, J = 6.70 Hz, |
| | | | 3H) |
| 25 | Ethyl-5-(4-hexyl-3- | Intermediate 19 | ¹H NMR (300 MHz, CDCl₃) |
| | propylphenyl)-6- | Benzaldehyde | δ 8.09 (d, J = 7.91 Hz, 1H), |
| | phenylpyridine-2- | CAS 100-52-7 | 7.83 (d, J = 7.91 Hz, 1H), |
| | carboxylate | | 7.38-7.42 (m, 2H), |
| | | | 7.19-7.24 (m, 3H), 7.07 (d, J = 7.91 Hz, |
| | | | 1H), 6.97 (dd, J = 2.05, |
| | | | 7.62 Hz, 1H), 6.89 (s, |
| | | | 1H), 4.48 (q, J = 7.23 Hz, |
| | | | 2H), 2.57 (t, J = 7.60 Hz, |
| | | | 2H), 2.45 (t, J = 7.80 Hz, |
| | | | 2H), 1.50-1.65 (m, 2H), |
| | | | 1.40-1.48 (m, J = 7.18, |
| | | | 7.18 Hz, 3H), |
| | | | 1.24-1.48 (m, 8H), 0.89 (t, J = 6.45 Hz, |
| | | | 3H), 0.82 (t, J = 7.33 Hz, |
| | | | 3H) |
| 26 | Ethyl-6-(3-chlorophenyl)- | Intermediate 19 | ¹H NMR (300 MHz, CDCl₃) |
| | 5-(4-hexyl-3-propyl | 3-chloro- | δ 8.12 (d, J = 8.20 Hz, 1H), |
| | phenyl)pyridine-2- | benzaldehyde | 7.85 (d, J = 7.91 Hz, 1H), |
| | carboxylate | CAS 587-04-2 | 7.45-7.47 (m, 1H), |
| | | | 7.19-7.24 (m, 2H), 7.13 (d, J = 7.33 Hz, |
| | | | 1H), 7.10 (d, J = 7.91 Hz, |
| | | | 1H), 6.96 (dd, J = 1.76, |
| | | | 7.91 Hz, 1H), 6.89 (d, |
| | | | J = 1.47 Hz, 1H), 4.50 (q, J = 7.13 Hz, |
| | | | 2H), 2.59 (t, J = 7.60 Hz, |
| | | | 2H), 2.49 (t, J = 7.60 Hz, |
| | | | 2H), |
| | | | 1.51-1.61 (m, 2H), 1.45 (d, J = 14.07 Hz, |
| | | | 3H), 1.26-1.48 (m, |
| | | | 8H), 0.86-0.92 (m, J = 6.70, |
| | | | 6.70 Hz, 3H), 0.85 (t, J = 7.00 Hz, |
| | | | 3H) |
| 27 | Ethyl-6-phenyl-5-[4-(3- | Intermediate 20 | ¹H NMR (300 MHz, CDCl₃) |
| | phenylpropyl)phenyl]pyridine- | Benzaldehyde | δ 8.11 (d, J = 7.91 Hz, 1H), |
| | 2-carboxylate | CAS 100-52-7 | 7.83 (d, J = 7.91 Hz, 1H), |
| | | | 7.38-7.42 (m, 2H), |
| | | | 7.15-7.31 (m, 8H), 7.09 (s, 4H), |
| | | | 4.49 (q, J = 7.23 Hz, 2H), |
| | | | 2.60-2.66 (m, 4H), |
| | | | 1.89-2.00 (m, 2H), 1.45 (t, J = 7.03 Hz, |
| | | | 3H) |
| 28 | Ethyl-6-(3-chlorophenyl)- | Intermediate 20 | ¹H NMR (300 MHz, CDCl₃) |
| | 5-[4-(3-phenylpropyl)phenyl]pyridine- | 3-chloro- | δ 8.13 (d, J = 7.91 Hz, 1H), |
| | 2- | benzaldehyde | 7.85 (d, J = 7.91 Hz, 1H), |
| | carboxylate | CAS 587-04-2 | 7.49 (t, J = 1.76 Hz, 1H), |
| | | | 7.07-7.31 (m, 12H), |
| | | | 4.50 (q, J = 7.23 Hz, 2H), |
| | | | 2.61-2.68 (m, 4H), |
| | | | 1.90-2.01 (m, 2H), 1.46 (t, J = 7.03 Hz, |
| | | | 3H) |
| 29 | Ethyl-5-(4-hexylphenyl)- | Intermediate 21 | ¹H NMR (300 MHz, CDCl₃) |
| | 6-(3-hydroxyphenyl) | 3-hydroxy- | δ 8.14 (d, J = 7.91 Hz, 1H), |
| | pyridine-2-carboxylate | benzaldehyde | 7.87 (d, J = 7.91 Hz, 1H), |
| | | CAS 100-83-4 | 7.20 (s, 1H), 7.06-7.13 (m, |
| | | | 4H), 6.92 (t, J = 7.62 Hz, |
| | | | 1H), 6.74 (br. s., 1H), |
| | | | 6.58-6.64 (m, 2H), 4.47 (q, J = 7.23 Hz, |
| | | | 2H), 2.58 (t, J = 7.91 Hz, |
| | | | 2H), |
| | | | 1.54-1.63 (m, 2H), 1.40 (t, J = 7.03 Hz, |
| | | | 3H), 1.24-1.34 (m, |
| | | | 6H), 0.88 (t, J = 6.74 Hz, |
| | | | 3H) |
| 30 | Ethyl-5-[4- | Intermediate 22 | ¹H NMR (300 MHz, CDCl₃) |
| | (pentyloxy)phenyl]-6- | Benzaldehyde | δ 8.10 (d, J = 7.91 Hz, 1H), |
| | phenylpyridine-2- | CAS 100-52-7 | 7.81 (d, J = 7.91 Hz, 1H), |
| | carboxylate | | 7.40-7.44 (m, 2H), |
| | | | 7.22-7.27 (m, 3H), |
| | | | 7.06-7.11 (m, 2H), 6.78-6.83 (m, 2H), |
| | | | 4.49 (q, J = 7.00 Hz, 2H), |
| | | | 3.93 (t, J = 6.59 Hz, 2H), |
| | | | 1.73-1.83 (m, 2H), 1.45 (t, |
| | | | J = 7.03 Hz, 3H), |
| | | | 1.31-1.49 (m, 4H), 0.93 (t, J = 7.33 Hz, |
| | | | 3H) |
| 31 | Ethyl-6-(4-fluorophenyl)- | Intermediate 22 | ¹H NMR (300 MHz, CDCl₃) |
| | 5-[4- | 4-fluoro- | δ 8.09 (d, J = 7.91 Hz, 1H), |
| | (pentyloxy)phenyl]pyridine- | benzaldehyde | 7.81 (d, J = 7.91 Hz, 1H), |
| | 2-carboxylate | CAS 459-57-4 | 7.40 (dd, J = 5.42, 8.94 Hz, |
| | | | 2H), 7.05-7.11 (m, 2H), |
| | | | 6.90-6.98 (m, 2H), |
| | | | 6.80-6.85 (m, 2H), 4.49 (q, J = 7.03 Hz, |
| | | | 2H), 3.95 (t, J = 6.59 Hz, |
| | | | 2H), |
| | | | 1.73-1.84 (m, 2H), 1.45 (t, J = 7.18 Hz, |
| | | | 3H), 1.34-1.47 (m, |
| | | | 4H), 0.94 (t, J = 7.30 Hz, |
| | | | 3H) |
| 32 | Ethyl-6-(3-fluorophenyl)- | Intermediate 22 | ¹H NMR (300 MHz, CDCl₃) |
| | 5-[4-(pentyloxy) | 3-fluoro- | δ 8.12 (d, J = 7.91 Hz, 1H), |
| | phenyl]pyridine-2- | benzaldehyde | 7.83 (d, J = 8.20 Hz, 1H), |
| | carboxylate | CAS 456-48-4 | 7.11-7.24 (m, 3H), 7.09 (d, |
| | | | J = 8.79 Hz, 1H), 7.09 (q, J = 4.98 Hz, |
| | | | 1H), |
| | | | 6.92-7.00 (m, 1H), 6.80-6.85 (m, 2H), |
| | | | 4.49 (q, J = 7.13 Hz, 2H), |
| | | | 3.95 (t, J = 6.59 Hz, 2H), |
| | | | 1.74-1.84 (m, 2H), 1.46 (t, |
| | | | J = 7.00 Hz, 3H), |
| | | | 1.32-1.50 (m, 4H), 0.93 (t, J = 6.70 Hz, |
| | | | 3H) |
| 33 | Ethyl-5-(4-hexylphenyl)- | Intermediate 21 | ¹H NMR (300 MHz, CDCl₃) |
| | 6-(2-thienyl)pyridine-2- | 2-Thiophene | δ 7.56 (d, J = 7.91 Hz, 1H), |
| | carboxylate | carboxaldehyde | 7.27 (d, J = 5.86 Hz, 1H), |
| | | CAS 98-03-3 | 7.23 (s, 4H), 7.11 (d, J = 7.62 Hz, |
| | | | 1H), |
| | | | 6.76-6.86 (m, 1H), 6.62 (d, J = 3.81 Hz, |
| | | | 1H), 4.81 (s, 2H), |
| | | | 3.92-4.07 (m, 1H), 2.68 (t, J = 7.77 Hz, |
| | | | 2H), |
| | | | 1.62-1.72 (m, 2H), 1.27-1.42 (m, 6H), |
| | | | 0.90 (t, J = 6.74 Hz, 3H) |
| 34 | Ethyl-6-(5-fluoro-2- | Intermediate 21 | ¹H NMR (300 MHz, CDCl₃) |
| | thienyl)-5-(4- | 5-fluoro-2- | δ 7.02-7.09 (m, 5H), |
| | hexylphenyl)pyridine-2- | Thiophenecarboxaldehyde | 6.98 (t, J = 3.96 Hz, 1H), |
| | carboxylate | CAS 29669-49-6 | 6.60 (dd, J = 2.78, 6.59 Hz, 1H), |
| | | | 6.36 (dd, J = 1.47, 4.10 Hz, |
| | | | 1H), 4.31 (q, J = 7.33 Hz, |
| | | | 2H), 2.53 (dd, J = 7.62, 8.20 Hz, |
| | | | 2H), 1.50-1.59 (m, |
| | | | 2H), 1.36 (t, J = 7.18 Hz, |
| | | | 3H), 1.23-1.33 (m, 6H), |
| | | | 0.83-0.91 (m, 3H) |
| 35 | Ethyl-5-(4-hexyl-3- | Intermediate 19 | ¹H NMR (300 MHz, CDCl₃) |
| | propylphenyl)-6-(2- | 2- | δ 7.98 (d, J = 7.62 Hz, 1H), |
| | thienyl)pyridine-2- | Thiophenecarboxaldehyde | 7.69 (d, J = 7.91 Hz, 1H), |
| | carboxylate | CAS 98-03-3 | 7.28 (d, J = 4.98 Hz, 1H), |
| | | | 7.21 (d, J = 7.33 Hz, 1H), |
| | | | 7.06-7.12 (m, 2H), |
| | | | 6.81 (dd, J = 3.81, 4.98 Hz, 1H), |
| | | | 6.75 (d, J = 3.52 Hz, 1H), |
| | | | 4.49 (q, J = 7.23 Hz, 2H), |
| | | | 2.68 (t, J = 7.62 Hz, 1H), |
| | | | 2.60 (dd, J = 7.33, 7.91 Hz, |
| | | | 2H), 1.48 (t, J = 7.03 Hz, |
| | | | 3H), 1.32-1.69 (m, 10H), |
| | | | 0.89-0.99 (m, 6H)\ |
| 36 | Ethyl-5-[4-(3- | Intermediate 20 | ¹H NMR (300 MHz, CDCl₃) |
| | phenylpropyl)phenyl]-6- | 2-Thiophenecarboxaldehyde | δ 7.99 (d, J = 7.91 Hz, 1H), |
| | (2-thienyl)pyridine-2- | CAS 98-03-3 | 7.67 (s, 1H), 7.17-7.33 (m, |
| | carboxylate | | 10H), 6.81 (t, J = 4.40 Hz, |
| | | | 1H), 6.72 (dd, J = 1.03, 3.66 Hz, |
| | | | 1H), 4.49 (q, J = 7.23 Hz, |
| | | | 2H), 2.71 (dt, J = 7.76, |
| | | | 11.13 Hz, 4H), 2.01 (quin, J = 7.69 Hz, |
| | | | 2H), 1.47 (t, J = 7.03 Hz, |
| | | | 3H) |
| 37 | Ethyl-5-(4-hexylphenyl)- | Intermediate 21 | ¹H NMR (300 MHz, CDCl₃) |
| | 6-(1,3-oxazol-4- | 4-Oxazolecarboxaldehyde | δ 8.11 (d, J = 7.91 Hz, 1H), |
| | yl)pyridine-2-carboxylate | CAS 118994-84-6 | 7.83 (s, 1H), 7.78 (d, J = 7.91 Hz, |
| | | | 1H), 7.31 (s, 1H), |
| | | | 7.17-7.27 (m, 4H), 4.51 (q, |
| | | | J = 7.23 Hz, 2H), 2.66 (t, J = 7.77 Hz, |
| | | | 2H), |
| | | | 1.60-1.70 (m, 2H), 1.47 (t, J = 7.18 Hz, |
| | | | 3H), 1.33 (br. s., 6H), |
| | | | 0.90 (t, J = 6.74 Hz, 3H) |
| 38 | Ethyl-5-(4-hexylphenyl)- | Intermediate 21 | ¹H NMR (300 MHz, CDCl₃) |
| | 6-(1,3-thiazol-2- | 2-Thiazole | δ 8.15 (d, J = 7.91 Hz, 1H), |
| | yl)pyridine-2-carboxylate | carboxaldehyde | 7.86 (d, J = 7.91 Hz, 1H), |
| | | CAS 10200-59-6 | 7.69 (d, J = 3.22 Hz, 1H), |
| | | | 7.37 (d, J = 3.22 Hz, 1H), |
| | | | 7.15-7.25 (m, 4H), 4.51 (q, |
| | | | J = 7.23 Hz, 2H), 2.65 (t, J = 7.77 Hz, |
| | | | 2H), |
| | | | 1.59-1.69 (m, 2H), 1.47 (t, J = 7.03 Hz, |
| | | | 3H), 1.22-1.39 (m, |
| | | | 6H), 0.89 (t, J = 6.30 Hz, |
| | | | 3H) |
| 39 | Ethyl-6-(2-furyl)-5-(4- | Intermediate 21 | ¹H NMR (300 MHz, CDCl₃) |
| | hexylphenyl)pyridine-2- | 2-Furancarboxaldehyde | δ 8.03 (d, J = 7.91 Hz, 1H), |
| | carboxylate | CAS 98-01-1 | 7.72 (d, J = 7.91 Hz, 1H), |
| | | | 7.42 (s, 1H), 7.18-7.26 (m, |
| | | | 4H), 6.28 (dd, J = 1.76, 3.22 Hz, |
| | | | 1H), 6.09 (d, J = 3.52 Hz, |
| | | | 1H), 4.51 (q, J = 7.03 Hz, |
| | | | 2H), 2.68 (t, J = 7.77 Hz, |
| | | | 2H), 1.58-1.72 (m, |
| | | | 2H), 1.48 (t, J = 7.18 Hz, |
| | | | 3H), 1.29-1.39 (m, 6H), |
| | | | 0.87-0.94 (m, 3H) |

### Example 13

### Intermediate 40

### [5-(4-hexylphenyl)-6-(3-thienyl)pyridin-2-yl]methanol

To a solution of Intermediate 23 (350 mg, 0.89 mmol) in dichloromethane (10 mL) at −78° C. was added DIBAL-H (1.0 M in dichloromethane, 4.5 mL). The reaction was warmed to RT over for 3 h with stirring and was quenched at −10° C. with ethyl acetate methanol, and 10% solution of HCl. The mixture was diluted with water. The aqueous layer was washed with ethyl acetate. The combined organic layers were washed with brine, dried over Na₂SO₄, and concentrated under reduced pressure. The residue was purified by MPLC (40% ethyl acetate in hexanes) to give 227 mg of the desired alcohol as colorless oil. ¹H NMR (300 MHz, CDCl₃) δ 7.64 (d, J=7.91 Hz, 1H), 7.25 (dd, J=1.32, 2.78 Hz, 1H), 7.11-7.19 (m, 5H), 7.13 (d, J=2.93 Hz, 1H), 7.09 (dd, J=1.17, 4.98 Hz, 1H), 4.82 (s, 2H), 2.64 (t, J=7.62 Hz, 2H), 1.58-1.69 (m, 2H), 1.32 (br. s., 6H), 0.90 (t, J=6.45 Hz, 3H)

Intermediates 41-61 were prepared from the corresponding starting materials, in a similar manner to the method described in Example 13 for Intermediate 40. The starting materials and the results are described below in Table 5.

TABLE 5

| Interm. | | | ¹H NMR δ (ppm) for |
|---|---|---|---|
| number | IUPAC name | starting material | Intermediate |
| 41 | (4-octyl-1,1′:2′,1″-terphenyl- | Intermediate 4 | ¹H NMR (300 MHz, |
| | 4′-yl)methanol | | CDCl₃) δ 7.39-7.46 (m, |
| | | | 3H), 7.14-7.24 (m, 5H), |
| | | | 7.04 (s, 4H), 4.77 (s, 2H), |
| | | | 2.57 (t, J = 7.62 Hz, 2H), |
| | | | 1.55-1.64 (m, 2H), |
| | | | 1.25-1.36 (m, 10H), |
| | | | 0.88-0.95 (m, 3H) |
| 42 | [6-(6-hexylpyridin-3- | Intermediate 5 | ¹H NMR (300 MHz, |
| | yl)biphenyl-3-yl]methanol | | CDCl₃) δ 8.03-8.06 (m, J = 0.59, |
| | | | 2.30 Hz, 1H), |
| | | | 7.42 (s, 2H), 7.35 (d, J = 7.91 Hz, |
| | | | 1H), 7.17-7.24 (m, |
| | | | 4H), 7.04-7.08 (m, 2H), |
| | | | 6.93 (d, J = 7.91 Hz, 1H), |
| | | | 4.77 (s, 2H), 2.73 (t, J = 7.30 Hz, |
| | | | 2H), |
| | | | 1.62-1.73 (m, 2H), 1.25-1.34 (m, |
| | | | 6H), 0.87 (t, J = 6.74 Hz, |
| | | | 3H) |
| 43 | [4-(3,3,4,4,5,5,6,6,6- | Intermediate 7 | ¹H NMR (300 MHz, |
| | nonafluorohexyl)-1,1′:2′,1″- | | CDCl₃) δ 7.42 (s, 3H), |
| | terphenyl-4′-yl]methanol | | 7.19-7.24 (m, 3H), |
| | | | 7.10-7.16 (m, 2H), |
| | | | 7.04-7.09 (m, 4H), 4.78 (s, 2H), |
| | | | 2.84-2.90 (m, 2H), |
| | | | 2.25-2.44 (m, 2H), 1.70 (br. s., 1H) |
| 44 | [5-(4-hexylphenyl)-6- | Intermediate 24 | ¹H NMR (300 MHz, |
| | phenylpyridin-2-yl]methanol | | CDCl₃) δ 7.71 (d, J = 7.62 Hz, |
| | | | 1H), 7.35-7.39 (m, |
| | | | 2H), 7.21-7.26 (m, 3H), |
| | | | 7.07 (s, 5H), 4.83 (s, 2H), |
| | | | 2.55-2.61 (m, 2H), |
| | | | 1.53-1.64 (m, 2H), 1.31 (s, 6H), |
| | | | 0.88 (t, J = 6.15 Hz, 3H) |
| 45 | 4-(3-phenylpropyl)-1,1′:2′,1″- | Intermediate 8 | ¹H NMR (300 MHz, |
| | terphenyl-4′-methanol | | CDCl₃) δ 7.37-7.44 (m, |
| | | | 3H), 7.24-7.30 (m, 2H), |
| | | | 7.11-7.20 (m, 7H), |
| | | | 6.99-7.05 (m, 4H), 4.75 (s, 2H), |
| | | | 2.60 (td, J = 3.81, 7.62 Hz, |
| | | | 4H), 1.87-1.98 (m, 2H), |
| | | | 1.76 (s, OH) |
| 46 | [5-(4-hexyl-3-propylphenyl)-6- | Intermediate 25 | ¹H NMR (300 MHz, |
| | phenylpyridin-2-yl]methanol | | CDCl₃) δ 7.72 (d, J = 7.91 Hz, |
| | | | 1H), 7.34-7.38 (m, |
| | | | 2H), 7.20-7.25 (m, 4H), |
| | | | 7.06 (d, J = 7.62 Hz, 1H), |
| | | | 6.95 (dd, J = 2.05, 7.33 Hz, |
| | | | 1H), 6.87 (d, J = 1.76 Hz, |
| | | | 1H), 4.82 (s, 2H), |
| | | | 2.57 (t, J = 7.90 Hz, 2H), |
| | | | 2.45 (t, J = 7.60 Hz, 2H), |
| | | | 1.50-1.61 (m, 2H), |
| | | | 1.26-1.44 (m, 8H), 0.89 (t, J = 6.40 Hz, |
| | | | 3H), 0.82 (t, J = 7.33 Hz, |
| | | | 3H) |
| 47 | [6-(6-octylpyridin-3- | Intermediate 6 | ¹H NMR (300 MHz, |
| | yl)biphenyl-3-yl]methanol | | CDCl₃) δ 8.14 (dd, J = 0.59, |
| | | | 2.34 Hz, 1H), |
| | | | 7.36-7.45 (m, 3H), |
| | | | 7.18-7.26 (m, 4H), 7.06-7.11 (m, |
| | | | 2H), 6.94 (d, J = 7.91 Hz, |
| | | | 1H), 4.78 (s, 2H), 2.84 (br. |
| | | | s., 1H), 2.73 (t, J = 7.60 Hz, |
| | | | 2H), 1.63-1.73 (m, |
| | | | 2H), 1.23-1.34 (m, 10H), |
| | | | 0.87 (t, J = 7.00 Hz, 3H) |
| 48 | [6-(3-chlorophenyl)-5-(4- | Intermediate 26 | ¹H NMR (300 MHz, |
| | hexyl-3-propylphenyl)pyridin- | | CDCl₃) δ 7.73 (d, J = 7.91 Hz, |
| | 2-yl]methanol | | 1H), 7.41 (t, J = 1.76 Hz, |
| | | | 1H), 7.27 (d, J = 7.91 Hz, |
| | | | 1H), 7.20 (tt, J = 2.10, |
| | | | 7.60 Hz, 2H), 7.13 (d, J = 7.62 Hz, |
| | | | 1H), 7.08 (d, J = 7.91 Hz, |
| | | | 1H), 6.94 (dd, J = 1.90, |
| | | | 7.77 Hz, 1H), |
| | | | 6.87 (d, J = 1.76 Hz, 1H), |
| | | | 4.83 (s, 2H), 2.59 (t, J = 7.60 Hz, |
| | | | 2H), 2.48 (t, J = 7.60 Hz, |
| | | | 2H), 1.50-1.61 (m, |
| | | | 2H), 1.25-1.48 (m, 8H), |
| | | | 0.86-0.93 (m, J = 6.40, |
| | | | 6.40 Hz, 3H), 0.84 (t, J = 7.30 Hz, |
| | | | 3H) |
| 49 | {6-(3-chlorophenyl)-5-[4-(3- | Intermediate 28 | ¹H NMR (300 MHz, |
| | phenylpropyl)phenyl]pyridin- | | CDCl₃) δ 7.74 (d, J = 7.91 Hz, |
| | 2-yl}methanol | | 1H), 7.45 (s, 1H), |
| | | | 7.06-7.31 (m, 13H), 4.85 (s, |
| | | | 2H), 2.61-2.68 (m, 4H), |
| | | | 1.90-2.01 (m, 2H) |
| 50 | 3-[3-(4-hexylphenyl)-6- | Intermediate 29 | ¹H NMR (300 MHz, |
| | (hydroxymethyl)pyridin-2- | | CDCl₃) δ 7.72 (d, J = 7.91 Hz, |
| | yl]phenol | | 1H), 7.27 (d, J = 7.91 Hz, |
| | | | 1H), 6.97-7.09 (m, |
| | | | 6H), 6.74 (d, J = 7.62 Hz, |
| | | | 2H), 4.83 (s, 2H), 2.58 (t, J = 7.77 Hz, |
| | | | 2H), |
| | | | 1.54-1.64 (m, 2H), 1.23-1.34 (m, |
| | | | 6H), 0.88 (t, J = 6.59 Hz, |
| | | | 3H) |
| 51 | 6-phenyl-5-[4-(3- | Intermediate 27 | ¹H NMR (300 MHz, |
| | phenylpropyl)phenyl]pyridine- | | CDCl₃) 7.72 (d, J = 7.62 Hz, |
| | 2-methanol | | 1H), 7.36-7.39 (m, |
| | | | 2H), 7.15-7.31 (m, 9H), |
| | | | 7.08 (s, 4H), 4.84 (s, 2H), |
| | | | 2.63 (t, J = 7.60 Hz, 4H), |
| | | | 1.94 (t, J = 7.62 Hz, 2H) |
| 52 | {5-[4-(pentyloxy)phenyl]-6- | Intermediate 30 | ¹H NMR (300 MHz, |
| | phenylpyridin-2-yl}methanol | | CDCl₃) δ 7.69 (d, J = 7.91 Hz, |
| | | | 1H), 7.35-7.41 (m, |
| | | | 2H), 7.21-7.27 (m, 4H), |
| | | | 7.03-7.09 (m, 2H), |
| | | | 6.76-6.82 (m, 2H), 4.82 (s, 2H), |
| | | | 3.92 (t, J = 6.59 Hz, 2H), |
| | | | 1.72-1.82 (m, 2H), |
| | | | 1.31-1.49 (m, 4H), 0.93 (t, J = 7.00 Hz, |
| | | | 3H) |
| 53 | {6-(4-fluorophenyl)-5-[4- | Intermediate 31 | ¹H NMR (300 MHz, |
| | (pentyloxy)phenyl]pyridin-2- | | CDCl₃) δ 7.69 (d, J = 7.91 Hz, |
| | yl}methanol | | 1H), 7.33-7.40 (m, |
| | | | 2H), 7.24 (d, J = 7.91 Hz, |
| | | | 1H), 7.03-7.09 (m, 2H), |
| | | | 6.90-6.98 (m, 2H), |
| | | | 6.78-6.84 (m, 2H), 4.82 (s, 2H), |
| | | | 3.94 (t, J = 6.59 Hz, 2H), |
| | | | 1.74-1.84 (m, 2H), |
| | | | 1.35-1.50 (m, 4H), 0.94 (t, J = 7.00 Hz, |
| | | | 3H) |
| 54 | {6-(3-fluorophenyl)-5-[4- | Intermediate 32 | ¹H NMR (300 MHz, |
| | (pentyloxy)phenyl]pyridin-2- | | CDCl₃) δ 7.71 (d, J = 7.91 Hz, |
| | yl}methanol | | 1H), 7.27 (d, J = 8.20 Hz, |
| | | | 1H), 7.09-7.24 (m, |
| | | | 3H), 7.07 (d, J = 8.79 Hz, |
| | | | 1H), 7.07 (q, J = 4.98 Hz, |
| | | | 1H), 6.96 (d, J = 1.17 Hz, |
| | | | 1H), 6.81 (d, J = 8.79 Hz, |
| | | | 1H), 6.81 (q, J = 5.00 Hz, |
| | | | 1H), 4.84 (s, 2H), 3.94 (t, J = 6.59 Hz, |
| | | | 2H), 3.86 (br. |
| | | | s., 1H), 1.74-1.84 (m, |
| | | | 2H), 1.32-1.50 (m, 4H), |
| | | | 0.93 (t, J = 7.00 Hz, 3H) |
| 55 | [5-(4-hexylphenyl)-6-(2- | Intermediate 33 | ¹H NMR (300 MHz, |
| | thienyl)pyridin-2-yl]methanol | | CDCl₃) δ 7.56 (d, J = 7.91 Hz, |
| | | | 1H), 7.27 (d, J = 5.86 Hz, |
| | | | 1H), 7.23 (s, 4H), |
| | | | 7.11 (d, J = 7.62 Hz, 1H), |
| | | | 6.76-6.86 (m, 1H), 6.62 (d, J = 3.81 Hz, |
| | | | 1H), 4.81 (s, 2H), |
| | | | 3.92-4.07 (m, 1H), |
| | | | 2.68 (t, J = 7.77 Hz, 2H), |
| | | | 1.62-1.72 (m, 2H), |
| | | | 1.27-1.42 (m, 6H), 0.90 (t, J = 6.74 Hz, |
| | | | 3H) |
| 56 | [6-(5-fluoro-2-thienyl)-5-(4- | Intermediate 34 | ¹H NMR (300 MHz, |
| | hexylphenyl)pyridin-2- | | CDCl₃) δ 7.51 (d, J = 7.91 Hz, |
| | yl]methanol | | 1H), 7.21-7.27 (m, |
| | | | 4H), 7.09 (d, J = 7.91 Hz, |
| | | | 1H), 6.16 (d, J = 2.64 Hz, |
| | | | 2H), 4.78 (s, 2H), 3.74 (br. |
| | | | s, 1H), 2.68 (t, J = 7.62 Hz, |
| | | | 2H), 1.62-1.72 (m, |
| | | | 2H), 1.27-1.39 (m, 6H), |
| | | | 0.90 (t, J = 6.45 Hz, 3H) |
| 57 | [5-(4-hexyl-3-propylphenyl)-6- | Intermediate 35 | ¹H NMR (600 MHz, |
| | (2-thienyl)pyridin-2- | | CDCl₃) δ 7.60 (d, J = 7.92 Hz, |
| | yl]methanol | | 1H), 7.24-7.29 (m, |
| | | | 1H), 7.20 (d, J = 7.63 Hz, |
| | | | 1H), 7.14 (d, J = 7.92 Hz, |
| | | | 1H), 7.04-7.10 (m, 2H), |
| | | | 6.82 (dd, J = 3.82, 4.99 Hz, |
| | | | 1H), 6.77 (d, J = 3.23 Hz, |
| | | | 1H), 4.82 (s, 2H), |
| | | | 3.41 (d, J = 6.46 Hz, 1H), |
| | | | 2.67 (dd, J = 7.90 Hz, 2H), |
| | | | 2.59 (dd, J = 7.34, 7.92 Hz, |
| | | | 2H), 1.53-1.65 (m, 4H), |
| | | | 1.33-1.44 (m, 4H), |
| | | | 0.90-0.95 (m, 6H) |
| 58 | {5-[4-(3- | Intermediate 36 | ¹H NMR (300 MHz, |
| | phenylpropyl)phenyl]-6-(2- | | CDCl₃) δ 7.54 (d, J = 7.91 Hz, |
| | thienyl)pyridin-2-yl}methanol | | 1H), 7.16-7.33 (m, |
| | | | 11H), 7.10 (d, J = 7.91 Hz, |
| | | | 1H), 6.79 (t, J = 4.40 Hz, |
| | | | 1H), 6.62 (d, J = 3.52 Hz, |
| | | | 1H), 4.79 (s, 2H), 4.04 (br. |
| | | | s, 1H), 2.66-2.75 (m, |
| | | | 4H), 1.95-2.08 (m, 2H) |
| 59 | [5-(4-hexylphenyl)-6-(1,3- | Intermediate 37 | ¹H NMR (600 MHz, |
| | oxazol-4-yl)pyridin-2- | | CDCl₃) δ 7.85 (d, J = 0.88 Hz, |
| | yl]methanol | | 1H), 7.62 (dd, J = 2.93, |
| | | | 7.92 Hz, 1H), |
| | | | 7.34 (d, J = 7.92 Hz, 1H), |
| | | | 7.22 (s, 2H), 7.14-7.19 (m, |
| | | | 2H), 6.98-7.01 (m, 1H), |
| | | | 4.90 (s, 2H), 4.52 (br. s, |
| | | | 1H), 2.66 (t, J = 7.78 Hz, |
| | | | 2H), 1.63-1.69 (m, 2H), |
| | | | 1.30-1.39 (m, 6H), |
| | | | 0.88-0.92 (m, 3H) |
| 60 | [5-(4-hexylphenyl)-6-(1,3- | Intermediate 38 | ¹H NMR (300 MHz, |
| | thiazol-2-yl)pyridin-2- | | CDCl₃) δ 7.64-7.68 (m, |
| | yl]methanol | | 2H), 7.33 (d, J = 7.91 Hz, |
| | | | 1H), 7.27 (dd, J = 1.90, |
| | | | 3.08 Hz, 1H), 7.16 (s, 4H), |
| | | | 4.83 (s, 2H), |
| | | | 2.59-2.65 (m, 2H), 1.57-1.67 (m, |
| | | | 2H), 1.19-1.37 (m, 6H), |
| | | | 0.87 (t, J = 6.45 Hz, 3H) |
| 61 | [6-(2-furyl)-5-(4- | Intermediate 39 | ¹H NMR (300 MHz, |
| | hexylphenyl)pyridin-2- | | CDCl₃) δ 7.59 (d, J = 7.91 Hz, |
| | yl]methanol | | 1H), 7.38 (s, 1H), |
| | | | 7.16-7.26 (m, 5H), 6.30 (dd, J = 1.76, |
| | | | 3.22 Hz, 1H), |
| | | | 6.10 (d, J = 3.22 Hz, 1H), |
| | | | 4.85 (s, 2H), 3.86 (br. s, 1H), |
| | | | 2.67 (t, J = 7.62 Hz, 2H), |
| | | | 1.61-1.71 (m, 2H), |
| | | | 1.27-1.42 (m, 6H), 0.90 (t, J = 7.03 Hz, |
| | | | 3H) |

### Example 14

### Intermediate 62

### 4-hexyl-1,1′:2′,1″-terphenyl-4′-carbaldehyde

To a vigorously stirred solution of pyridinium chlorochromate (1.29 g, 5.97 mmol) and celite (2.6 g) in dichloromethane (30 mL) was added a solution of (4-hexyl-1,1′:2′,1″-terphenyl-4′-yl)methanol (1.37 g, 3.98 mmol) in dichloromethane. After stirring at RT for 3 h, the reaction mixture was filtered through a plug of silica gel and eluted well with dichloromethane. Concentration yielded 1.21 g of the aldehyde as colorless oil.

¹H NMR (300 MHz, CDCl₃) δ 10.08 (s, 1H), 7.92 (s, 1H), 7.90 (dd, J=1.80, 7.33 Hz, 1H), 7.57-7.60 (m, 1H), 7.21-7.26 (m, 3H), 7.13-7.17 (m, 2H), 7.05 (s, 4H), 2.54-2.59 (m, 2H), 1.58 (s, 2H), 1.25-1.33 (m, 6H), 0.88 (t, J=6.74 Hz, 3H).

### Example 15

### Intermediate 63

### 5-(4-hexylphenyl)-6-(3-thienyl)pyridine-2-carbaldehyde

To a solution of Intermediate 40 (70 mg, 0.2 mmol), NMO (58 mg, 0.5 mmol), and 4 Å molecular sieves (140 mg) in dichloromethane (5 mL) and acetonitrile (0.6 mL) was added tetrapropylammonium perruthenate (TPAP, 4 mg). After stirring at RT for 2 h, the reaction mixture was filtered through a short column of silica gel, eluted with ethyl acetate and concentrated under reduced pressure. Purification by MPLC (10% ethyl acetate in hexanes) gave rise to 45 mg 5-(4-hexylphenyl)-6-(3-thienyl)pyridine-2-carbaldehyde as a yellow oil.

¹H NMR (300 MHz, CDCl₃) δ 10.15 (s, 1H), 7.91 (d, J=8.20 Hz, 1H), 7.80 (d, J=7.91 Hz, 1H), 7.33 (dd, J=1.17, 2.93 Hz, 1H), 7.12-7.22 (m, 6H), 2.65 (t, J=7.77 Hz, 2H), 1.59-1.69 (m, 2H), 1.25-1.41 (m, 6H), 0.90 (t, J=6.45 Hz, 3H).

Intermediates 64-84 were prepared from the corresponding starting materials, in a similar manner to the method described in Example 14 for Intermediate 62 or Example 15 for Intermediate 63. The starting materials and the results are described below in Table 6.

TABLE 6

| Intermediate | | | ¹H NMR δ (ppm) for |
|---|---|---|---|
| number | IUPAC name | starting material | Intermediate |
| 65 | 5-(4-hexylphenyl)-6- | Intermediate 44 | ¹H NMR (300 MHz, |
| | phenylpyridine-2- | | CDCl₃) δ 10.18 (d, J = 0.88 Hz, |
| | carbaldehyde | | 1H), 7.98 (d, J = 8.20 Hz, |
| | | | 1H), 7.89 (dd, J = 0.88, |
| | | | 7.91 Hz, 1H), |
| | | | 7.40-7.43 (m, 2H), |
| | | | 7.25-7.31 (m, 3H), 7.10 (s, 4H), |
| | | | 2.59 (dd, J = 7.60 Hz, 2H), |
| | | | 1.55-1.65 (m, 2H), |
| | | | 1.25-1.34 (m, 6H), 0.88 (t, J = 6.70 Hz, |
| | | | 3H) |
| 66 | 6-(6-hexylpyridin-2- | Intermediate 42 | ¹H NMR (300 MHz, |
| | yl)biphenyl-3- | | CDCl₃) δ 10.11 (s, 1H), |
| | carbaldehyde | | 8.39 (dd, J = 0.88, 2.34 Hz, |
| | | | 1H), 7.93-7.97 (m, |
| | | | 2H), 7.59 (d, J = 8.50 Hz, |
| | | | 1H), 7.25-7.31 (m, 4H), |
| | | | 7.12-7.16 (m, 2H), |
| | | | 7.00 (dd, J = 0.59, 8.20 Hz, |
| | | | 1H), 2.76 (t, J = 8.00 Hz, |
| | | | 2H), 1.60-1.75 (m, 2H), |
| | | | 1.26-1.35 (m, 6H), |
| | | | 0.88 (t, J = 6.74 Hz, 3H). |
| 67 | 4-(3-phenylpropyl)- | Intermediate 45 | ¹H NMR (300 MHz, CDCl₃) |
| | 1,1′:2′,1″-terphenyl-4′- | | δ 10.08 (s, 1H), |
| | carbaldehyde | | 7.88-7.92 (m, 2H), 7.58 (d, J = 8.50 Hz, |
| | | | 1H), |
| | | | 7.13-7.30 (m, 10H), 7.05 (s, 4H), |
| | | | 2.61 (t, J = 7.62 Hz, 4H), |
| | | | 1.88-1.99 (m, 2H). |
| 68 | 6-phenyl-5-[4-(3- | Intermediate 51 | ¹H NMR (300 MHz, |
| | phenylpropyl)phenyl]pyridine- | | CDCl₃) δ 10.18 (s, 1H), |
| | 2-carbaldehyde | | 7.97-8.00 (m, 1H), |
| | | | 7.89 (d, J = 7.91 Hz, 1H), |
| | | | 7.39-7.43 (m, 2H), |
| | | | 7.25-7.31 (m, 4H), 7.15-7.21 (m, |
| | | | 3H), 7.11 (s, 4H), 2.64 (td, |
| | | | J = 2.93, 7.90 Hz, 4H), |
| | | | 1.90-2.00 (m, 2H) |
| 69 | 5-(4-hexylphenyl)-6-(3- | Intermediate 50 | ¹H NMR (300 MHz, |
| | hydroxyphenyl)pyridine-2- | | CDCl₃) δ 10.18 (s, 1H), |
| | carbaldehyde | | 7.99 (d, J = 8.20 Hz, 1H), |
| | | | 7.90 (d, J = 7.62 Hz, 1H), |
| | | | 7.01-7.13 (m, 6H), |
| | | | 6.82-6.85 (m, 1H), 6.76 (ddd, J = 1.20, |
| | | | 2.34, 8.20 Hz, 1H), |
| | | | 2.60 (t, J = 7.90 Hz, 2H), |
| | | | 1.59 (d, J = 7.33 Hz, 2H), |
| | | | 1.25-1.34 (m, 6H), |
| | | | 0.88 (t, J = 6.45 Hz, 3H) |
| 70 | 5-(4-hexyl-3- | Intermediate 46 | ¹H NMR (300 MHz, |
| | propylphenyl)-6- | | CDCl₃) δ 10.18 (d, J = 0.59 Hz, |
| | phenylpyridine-2- | | 2H), 7.97 (d, J = 7.91 Hz, |
| | carbaldehyde | | 1H), 7.89 (d, J = 7.62 Hz, |
| | | | 1H), |
| | | | 7.39-7.43 (m, 2H), 7.25-7.30 (m, |
| | | | 3H), 7.07-7.11 (m, 1H), |
| | | | 6.99 (dd, J = 2.05, 7.91 Hz, |
| | | | 1H), 6.91 (d, J = 1.76 Hz, |
| | | | 1H), 2.58 (t, J = 7.90 Hz, |
| | | | 2H), 2.46 (t, J = 7.60 Hz, |
| | | | 2H), 1.50-1.62 (m, |
| | | | 2H), 1.26-1.45 (m, 8H), |
| | | | 0.90 (t, J = 6.70 Hz, 3H), |
| | | | 0.83 (t, J = 7.33 Hz, 3H) |
| 71 | 6-(3-chlorophenyl)-5-(4- | Intermediate 48 | ¹H NMR (300 MHz, |
| | hexyl-3- | | CDCl₃) δ 10.16 (s, 1H), |
| | propylphenyl)pyridine-2- | | 7.98 (d, J = 7.91 Hz, 1H), |
| | carbaldehyde | | 7.90 (d, J = 8.21 Hz, 1H), |
| | | | 7.47 (t, J = 1.76 Hz, 1H), |
| | | | 7.24 (tt, J = 1.47, 7.60 Hz, |
| | | | 2H), 7.17 (d, J = 7.62 Hz, |
| | | | 1H), 7.11 (d, J = 7.91 Hz, |
| | | | 1H), 6.98 (dd, J = 1.47, |
| | | | 7.62 Hz, 1H), 6.91 (d, J = 1.76 Hz, |
| | | | 1H), 2.60 (t, J = 7.60 Hz, |
| | | | 2H), 2.49 (t, J = 7.33 Hz, |
| | | | 2H), |
| | | | 1.51-1.62 (m, 2H), 1.28-1.49 (m, |
| | | | 8H), 0.86-0.93 (m, J = 6.70, |
| | | | 6.70 Hz, 3H), 0.85 (t, |
| | | | J = 7.30 Hz, 3H) |
| 72 | 6-(3-chlorophenyl)-5-[4-(3- | Intermediate 49 | ¹H NMR (300 MHz, |
| | phenylpropyl)phenyl]pyridine- | | CDCl₃) δ 10.17 (s, 3H), |
| | 2-carbaldehyde | | 8.00 (d, J = 7.91 Hz, 1H), |
| | | | 7.90 (d, J = 7.91 Hz, 1H), |
| | | | 7.50 (s, 1H), |
| | | | 7.26-7.31 (m, 3H), 7.09-7.22 (m, |
| | | | 9H), 2.65 (dt, J = 6.70, |
| | | | 7.33 Hz, 4H), 1.96 (quin, J = 7.62 Hz, |
| | | | 2H) |
| 73 | 4-(3,3,4,4,5,5,6,6,6- | Intermediate 43 | ¹H NMR (300 MHz, |
| | nonafluorohexyl)- | | CDCl₃) δ 10.09 (s, 1H), |
| | 1,1′:2′,1″-terphenyl-4′- | | 7.91-7.98 (m, 1H), |
| | carbaldehyde | | 7.91 (dd, J = 1.50, 6.45 Hz, |
| | | | 1H), 7.57 (d, J = 8.50 Hz, |
| | | | 1H), 7.22-7.27 (m, 3H), |
| | | | 7.12-7.17 (m, 2H), |
| | | | 7.07-7.11 (m, 4H), |
| | | | 2.86-2.91 (m, 2H), 2.26-2.45 (m, |
| | | | 2H) |
| 74 | 5-[4-(pentyloxy)phenyl]-6- | Intermediate 52 | ¹H NMR (300 MHz, |
| | phenylpyridine-2- | | CDCl₃) δ 10.17 (d, J = 0.59 Hz, |
| | carbaldehyde | | 1H), 7.97 (d, J = 7.62 Hz, |
| | | | 1H), 7.87 (dd, J = 0.88, |
| | | | 7.91 Hz, 1H), |
| | | | 7.40-7.46 (m, 2H), |
| | | | 7.28-7.33 (m, 3H), 7.08-7.13 (m, |
| | | | 2H), 6.79-6.84 (m, 2H), |
| | | | 3.94 (t, J = 6.59 Hz, 2H), |
| | | | 1.73-1.83 (m, 2H), |
| | | | 1.33-1.49 (m, 4H), 0.93 (t, J = 7.30 Hz, |
| | | | 3H) |
| 75 | 6-(3-fluorophenyl)-5-[4- | Intermediate 54 | ¹H NMR (300 MHz, |
| | (pentyloxy)phenyl]pyridine- | | CDCl₃) δ 10.17 (d, J = 0.60 Hz, |
| | 2-carbaldehyde | | 1H), 8.00 (d, J = 7.91 Hz, |
| | | | 1H), 7.90 (dd, J = 0.60, |
| | | | 7.90 Hz, 1H), |
| | | | 7.10-7.30 (m, 5H), |
| | | | 6.99-7.06 (m, 1H), 6.81-6.88 (m, |
| | | | 2H), 3.97 (t, J = 6.59 Hz, |
| | | | 2H), 1.81 (tdd, J = 6.74, |
| | | | 6.96, 7.07 Hz, 2H), |
| | | | 1.34-1.52 (m, 4H), |
| | | | 0.90-1.01 (m, 3H) |
| 76 | 6-(4-fluorophenyl)-5-[4- | Intermediate 53 | ¹H NMR (300 MHz, |
| | (pentyloxy)phenyl]pyridine- | | CDCl₃) δ 10.14 (d, J = 0.59 Hz, |
| | 2-carbaldehyde | | 1H), 7.95 (d, J = 7.91 Hz, |
| | | | 1H), 7.85 (dd, J = 0.88, |
| | | | 7.62 Hz, 1H), |
| | | | 7.38-7.45 (m, 2H), |
| | | | 7.07-7.12 (m, 2H), 6.94-7.02 (m, |
| | | | 2H), 6.80-6.86 (m, 2H), |
| | | | 3.95 (t, J = 6.59 Hz, 2H), |
| | | | 1.73-1.84 (m, 2H), |
| | | | 1.34-1.50 (m, 4H), 0.93 (t, J = 7.00 Hz, |
| | | | 3H) |
| 77 | 4-octyl-1,1′:2′,1″- | Intermediate 41 | ¹H NMR (300 MHz, |
| | terphenyl-4′-carbaldehyde | | CDCl₃) δ 10.08 (s, 1H), |
| | | | 7.91-7.93 (m, 1H), |
| | | | 7.90 (dd, J = 1.47, 7.03 Hz, |
| | | | 1H), 7.59 (d, J = 8.50 Hz, |
| | | | 1H), 7.22-7.26 (m, 3H), |
| | | | 7.13-7.17 (m, 2H), |
| | | | 7.05 (s, 4H), 2.53-2.59 (m, |
| | | | 2H), 1.55 (s, 2H), |
| | | | 1.24-1.32 (m, 10H), 0.88 (t, J = 6.74 Hz, |
| | | | 3H) |
| 78 | 5-(4-hexylphenyl)-6-(2- | Intermediate 55 | ¹H NMR (300 MHz, |
| | thienyl)pyridine-2- | | CDCl₃) δ 10.10 (s, 1H), |
| | carbaldehyde | | 7.80 (d, J = 7.62 Hz, 1H), |
| | | | 7.68 (d, J = 7.62 Hz, 1H), |
| | | | 7.28 (d, J = 4.98 Hz, 1H), |
| | | | 7.21 (s, 4H), 6.80 (t, J = 4.40 Hz, |
| | | | 1H), 6.68 (d, J = 3.52 Hz, |
| | | | 1H), 2.65 (t, J = 7.62 Hz, |
| | | | 2H), 1.63 (s, 2H), |
| | | | 1.23-1.39 (m, 6H), |
| | | | 0.87 (t, J = 6.30 Hz, 3H) |
| 79 | 6-(5-fluoro-2-thienyl)-5-(4- | Intermediate 56 | ¹H NMR (600 MHz, |
| | hexylphenyl)pyridine-2- | | CDCl₃) δ 10.09 (d, J = 0.59 Hz, |
| | carbaldehyde | | 1H), 7.81 (d, J = 7.92 Hz, |
| | | | 1H), 7.67 (dd, J = 0.88, |
| | | | 7.92 Hz, 1H), |
| | | | 7.24-7.29 (m, 4H), 6.26 (dd, J = 3.67, |
| | | | 4.26 Hz, 1H), |
| | | | 6.19 (dd, J = 1.91, 4.26 Hz, |
| | | | 1H), 2.69 (t, J = 7.34 Hz, |
| | | | 2H), 1.68 (quin, J = 7.56 Hz, |
| | | | 2H), 1.31-1.40 (m, |
| | | | 6H), 0.90 (t, J = 7.34 Hz, |
| | | | 3H) |
| 80 | 5-(4-hexyl-3- | Intermediate 57 | ¹H NMR (300 MHz, |
| | propylphenyl)-6-(2- | | CDCl₃) δ 10.10 (s, 1H), |
| | thienyl)pyridine-2- | | 7.81 (d, J = 7.91 Hz, 1H), |
| | carbaldehyde | | 7.69 (d, J = 7.62 Hz, 1H), |
| | | | 7.28 (d, J = 4.98 Hz, 1H), |
| | | | 7.19 (d, J = 7.62 Hz, 1H), |
| | | | 7.04-7.09 (m, 2H), |
| | | | 6.80 (t, J = 4.40 Hz, 1H), |
| | | | 6.70 (d, J = 3.81 Hz, 1H), |
| | | | 2.65 (dd, J = 6.74, 7.91 Hz, |
| | | | 2H), 2.58 (dd, J = 7.62, |
| | | | 8.50 Hz, 2H), 1.57 (s, 4H), |
| | | | 1.24-1.44 (m, 6H), |
| | | | 0.84-0.95 (m, 6H) |
| 81 | 5-[4-(3- | Intermediate 58 | ¹H NMR (600 MHz, |
| | phenylpropyl)phenyl]-6- | | CDCl₃) δ 10.15 (d, J = 0.59 Hz, |
| | (2-thienyl)pyridine-2- | | 1H), 7.86 (d, J = 7.63 Hz, |
| | carbaldehyde | | 1H), 7.73 (dd, J = 0.73, |
| | | | 7.78 Hz, 1H), |
| | | | 7.20-7.33 (m, 10H), 6.85 (dd, J = 3.81, |
| | | | 4.99 Hz, 1H), |
| | | | 6.74 (dd, J = 0.88, 3.81 Hz, |
| | | | 1H), 2.75 (t, J = 7.92 Hz, |
| | | | 2H), 2.71 (t, J = 7.63 Hz, |
| | | | 2H), 2.01-2.06 (m, 2H) |
| 82 | 5-(4-hexylphenyl)-6-(1,3- | Intermediate 59 | ¹H NMR (300 MHz, |
| | oxazol-4-yl)pyridine-2- | | CDCl₃) δ 10.26 (s, 1H), |
| | carbaldehyde | | 7.97 (d, J = 7.62 Hz, 1H), |
| | | | 7.90 (s, 1H), 7.81 (d, J = 7.91 Hz, |
| | | | 1H), |
| | | | 7.19-7.29 (m, 4H), 7.07 (s, 1H), |
| | | | 2.68 (t, J = 7.77 Hz, 2H), |
| | | | 1.61-1.72 (m, 2H), |
| | | | 1.22-1.41 (m, 6H), 0.83-0.95 (m, |
| | | | 3H) |
| 83 | 5-(4-hexylphenyl)-6-(1,3- | Intermediate 60 | ¹H NMR (300 MHz, |
| | thiazol-2-yl)pyridine-2- | | CDCl₃) δ 10.19 (s, 1H), |
| | carbaldehyde | | 7.98-8.07 (m, J = 8.50 Hz, |
| | | | 1H), 7.90 (d, J = 8.21 Hz, |
| | | | 1H), 7.75 (d, J = 2.93 Hz, |
| | | | 1H), 7.39 (d, J = 3.22 Hz, |
| | | | 1H), 7.19-7.24 (m, |
| | | | 4H), 2.66 (t, J = 7.62 Hz, |
| | | | 2H), 1.58-1.70 (m, 2H), |
| | | | 1.25-1.40 (m, 6H), |
| | | | 0.90 (t, J = 6.01 Hz, 3H) |
| 84 | 6-(2-furyl)-5-(4- | Intermediate 61 | ¹H NMR (300 MHz, |
| | hexylphenyl)pyridine-2- | | CDCl₃) δ 10.20 (s, 1H), |
| | carbaldehyde | | 7.89 (d, J = 7.62 Hz, 1H), |
| | | | 7.76 (d, J = 7.91 Hz, 1H), |
| | | | 7.46 (s, 1H), |
| | | | 7.19-7.27 (m, 4H), 6.33 (dd, J = 1.61, |
| | | | 3.37 Hz, 1H), |
| | | | 6.12 (d, J = 3.22 Hz, 1H), |
| | | | 2.68 (t, J = 7.62 Hz, 2H), |
| | | | 1.61-1.73 (m, 2H), |
| | | | 1.25-1.42 (m, 6H), 0.90 (t, J = 6.30 Hz, |
| | | | 3H) |

### Example 16

### Compound 1

### (3-{[6-(5-Hexyl-pyridin-2-yl)-biphenyl-3-ylmethyl]-amino}-propyl)-phosphonic acid

To a solution of 6-(5-hexylpyridin-2-yl)biphenyl-3-carbaldehyde (80 mg, 0.233 mmol) and (3-aminopropyl)phosphonic acid (32.4 mg) in methanol was added tetrabutylammonium hydroxide (1M in MeOH, 0.23 mL). The reaction mixture was heated to 50° C. for 30 min with stirring, then sodium cyanoborohydride (41 mg, 0.65 mmol) was added. The reaction mixture was heated to 50° C. with stirring for 3 h. After cooling to RT, the mixture was concentrated and purified by MPLC (0-100% ethyl acetate in hexanes) to give 36 mg of the desired product as a colorless solid. ¹H NMR (300 MHz, CD₃OD) δ 8.34 (d, J=1.76 Hz, 1H), 7.58-7.63 (m, 3H), 7.41 (dd, J=2.34, 8.20 Hz, 1H), 7.20-7.25 (m, 3H), 7.11-7.16 (m, 2H), 6.91 (d, J=7.91 Hz, 1H), 4.21 (s, 2H), 3.11 (t, J=6.30 Hz, 2H), 2.61 (t, J=7.62 Hz, 2H), 1.92-2.07 (m, 2H), 1.55-1.74 (m, 4H), 1.31 (br. s., 6H), 0.86-0.92 (m, 3H).

Compounds 2-28 were prepared from the corresponding starting materials, in a similar manner to the method described in Example 16 for Compound 1. The starting materials and the results are described below in Table 7.

TABLE 7

| Comp. | | starting | |
|---|---|---|---|
| number | IUPAC name | material | ¹H NMR δ (ppm) for Compound |
| 2 | (3-{[5-(4-Hexyl-phenyl)-6- | Intermediate | ¹H NMR (300 MHz, CD₃OD) δ 7.66 (d, |
| | thiophen-2-yl-pyridin-2- | 78 | J = 7.91 Hz, 1H), 7.35-7.39 (m, 2H), |
| | ylmethyl]-amino}-propyl)- | | 7.17-7.27 (m, 4H), 6.80 (t, J = 4.25 Hz, |
| | phosphonic acid | | 1H), 6.68 (d, J = 3.81 Hz, 1H), |
| | | | 4.30 (s, 2H), 3.17-3.26 (m, 2H), |
| | | | 2.68 (t, J = 7.62 Hz, 2H), 1.98-2.12 (m, |
| | | | 2H), 1.61-1.77 (m, 4H), |
| | | | 1.28-1.48 (m, 6H), 0.91 (t, J = 6.74 Hz, 3H) |
| 3 | (3-{[6-(5-Fluoro-thiophen- | Intermediate | ¹H NMR (300 MHz, CD₃OD) δ 7.68 (d, |
| | 2-yl)-5-(4-hexyl-phenyl)- | 79 | J = 7.91 Hz, 1H), 7.41 (d, J = 7.91 Hz, |
| | pyridin-2-ylmethyl]- | | 1H), 7.26-7.37 (m, 4H), 6.35 (dd, J = 3.80 Hz, |
| | amino}-propyl)- | | 1H), 6.30 (dd, J = 1.76, |
| | phosphonic acid | | 4.10 Hz, 1H), 4.36 (s, 2H), 3.26 (t, J = 6.15 Hz, |
| | | | 2H), 2.75 (t, J = 7.47 Hz, 2H), |
| | | | 2.03-2.18 (m, 2H), 1.66-1.82 (m, |
| | | | 4H), 1.37-1.53 (m, 6H), 0.96 (t, J = 6.74 Hz, |
| | | | 3H) |
| 4 | (3-{[5-(4-Hexyl-3-propyl- | Intermediate | ¹H NMR (300 MHz, CD₃OD) δ 7.64 (d, |
| | phenyl)-6-thiophen-2-yl- | 80 | J = 7.62 Hz, 1H), 7.34-7.38 (m, 2H), |
| | pyridin-2-ylmethyl]- | | 7.21 (d, J = 8.50 Hz, 1H), 7.05 (br. s., |
| | amino}-propyl)- | | 2H), 6.79 (t, J = 4.40 Hz, 1H), 6.67 (d, |
| | phosphonic acid | | J = 3.52 Hz, 1H), 4.23 (s, 2H), 3.12 (t, |
| | | | J = 6.30 Hz, 2H), 2.69 (t, J = 7.77 Hz, |
| | | | 2H), 2.60 (t, J = 7.47 Hz, 2H), |
| | | | 1.95-2.09 (m, 2H), 1.52-1.75 (m, 6H), |
| | | | 1.30-1.48 (m, 6H), 0.93 (t, J = 7.03 Hz, |
| | | | 6H) |
| 5 | [3-({5-[4-(3-Phenyl- | Intermediate | ¹H NMR (300 MHz, CD₃OD) δ 7.68 (d, |
| | propyl)-phenyl]-6- | 81 | J = 7.91 Hz, 1H), 7.35-7.39 (m, 2H), |
| | thiophen-2-yl-pyridin-2- | | 7.15-7.30 (m, 9H), 6.81 (t, J = 4.54 Hz, |
| | ylmethyl}-amino)-propyl]- | | 1H), 6.70 (d, J = 3.81 Hz, 1H), |
| | phosphonic acid | | 4.29 (s, 2H), 3.19 (t, J = 6.74 Hz, 2H), |
| | | | 2.63-2.75 (m, 4H), 1.95-2.09 (m, |
| | | | 4H), 1.66-1.77 (m, 2H) |
| 6 | (3-{[5-(4-Hexyl-phenyl)-6- | Intermediate | ¹H NMR (300 MHz, CD₃OD) δ 7.74 (d, |
| | thiophen-3-yl-pyridin-2- | 63 | J = 7.91 Hz, 1H), 7.42 (d, J = 8.21 Hz, |
| | ylmethyl]-amino}-propyl)- | | 1H), 7.40 (d, J = 2.93 Hz, 1H), |
| | phosphonic acid | | 7.11-7.23 (m, 5H), 7.05 (d, J = 4.98 Hz, |
| | | | 1H), 4.31 (s, 2H), 3.16 (t, J = 6.45 Hz, |
| | | | 2H), 2.64 (t, J = 7.62 Hz, 2H), 2.03 (dt, |
| | | | J = 6.78, 16.92 Hz, 2H), |
| | | | 1.58-1.76 (m, 4H), 1.29-1.45 (m, 6H), 0.90 (t, J = 6.15 Hz, |
| | | | 3H) |
| 7 | (3-{[5-(4-Hexyl-phenyl)-6- | Intermediate | ¹H NMR (600 MHz, CD₃OD) δ 8.31 (d, |
| | oxazol-4-yl-pyridin-2- | 82 | J = 0.88 Hz, 1H), 7.75 (d, J = 7.63 Hz, |
| | ylmethyl]-amino}-propyl)- | | 1H), 7.49 (d, J = 7.63 Hz, 1H), 7.33 (d, |
| | phosphonic acid | | J = 8.22 Hz, 2H), 7.20 (d, J = 7.92 Hz, |
| | | | 2H), 6.88 (s, 1H), 4.39 (s, 2H), 3.19 (t, |
| | | | J = 6.75 Hz, 2H), 2.71 (t, J = 7.63 Hz, |
| | | | 2H), 2.07 (ddtd, J = 6.46, 7.04, 7.63, |
| | | | 17.02 Hz, 2H), 1.63-1.75 (m, 4H), |
| | | | 1.32-1.45 (m, 6H), 0.91 (t, J = 7.04 Hz, |
| | | | 3H) |
| 8 | (3-{[5-(4-Hexyl-phenyl)-6- | Intermediate | ¹H NMR (600 MHz, CD₃OD) δ 7.83 (d, |
| | thiazol-2-yl-pyridin-2- | 83 | J = 7.92 Hz, 1H), 7.74 (d, J = 3.23 Hz, |
| | ylmethyl]-amino}-propyl)- | | 1H), 7.60 (s, 1H), 7.58 (d, J = 7.92 Hz, |
| | phosphonic acid | | 1H), 7.21 (d, J = 8.22 Hz, 2H), 7.17 (d, |
| | | | J = 7.92 Hz, 2H), 4.24 (s, 2H), 3.05 (t, |
| | | | J = 6.60 Hz, 2H), 2.65 (dd, J = 7.60 Hz, |
| | | | 1H), 1.96-2.03 (m, 2H), |
| | | | 1.62-1.71 (m, 4H), 1.29-1.45 (m, 6H), |
| | | | 0.89-0.93 (m, 3H) |
| 9 | (3-{[6-Furan-2-yl-5-(4- | Intermediate | ¹H NMR (300 MHz, CD₃OD) δ 7.70 (d, |
| | hexyl-phenyl)-pyridin-2- | 84 | J = 7.91 Hz, 1H), 7.45 (s, 2H), 7.24 (d, |
| | ylmethyl]-amino}-propyl)- | | J = 7.62 Hz, 2H), 7.15 (d, J = 8.20 Hz, |
| | phosphonic acid | | 2H), 6.35-6.38 (m, J = 1.76 Hz, 1H), |
| | | | 6.26 (d, J = 2.93 Hz, 1H), 4.34 (s, 2H), |
| | | | 3.20 (t, J = 6.30 Hz, 2H), 2.67 (t, J = 7.62 Hz, |
| | | | 2H), 1.98-2.12 (m, 2H), |
| | | | 1.61-1.78 (m, 4H), 1.27-1.46 (m, |
| | | | 6H), 0.91 (t, J = 6.01 Hz, 3H) |
| 10 | (3-{[5-(4-Hexyl-phenyl)-6- | Intermediate | ¹H NMR (300 MHz, CD₃OD) δ 7.83 (d, |
| | (3-hydroxy-phenyl)- | 69 | J = 7.91 Hz, 1H), 7.48 (d, J = 7.91 Hz, |
| | pyridin-2-ylmethyl]- | | 1H), 7.10 (s, 4H), 7.02 (s, 1H), |
| | amino}-propyl)- | | 6.98 (d, J = 7.62 Hz, 1H), 6.68 (d, J = 8.20 Hz, |
| | phosphonic acid | | 2H), 4.34 (s, 2H), 3.17-3.23 (m, |
| | | | 2H), 2.59 (t, J = 7.62 Hz, 2H), |
| | | | 1.96-2.09 (m, 2H), 1.55-1.76 (m, 4H), |
| | | | 1.27-1.48 (m, 6H), 1.02 (t, J = 7.33 Hz, |
| | | | 3H) |
| 11 | (3-{[4-(3,3,4,4,5,5,6,6,6- | Intermediate | ¹H NMR (300 MHz, CD₃OD) δ |
| | Nonafluoro-hexyl)- | 73 | 7.52-7.58 (m, 2H), 7.45 (d, J = 7.33 Hz, |
| | [1,1′;2′,1″]terphenyl-4′- | | 1H), 7.05-7.22 (m, 9H), 4.18 (s, 2H), |
| | ylmethyl]-amino}-propyl)- | | 3.12 (t, J = 6.15 Hz, 2H), |
| | phosphonic acid | | 2.84-2.91 (m, 2H), 2.33-2.52 (m, 2H), |
| | | | 1.92-2.07 (m, 2H), 1.61-1.74 (m, 2H) |
| 12 | (3-{[4-(3-Phenyl-propyl)- | Intermediate | ¹H NMR (300 MHz, CD₃OD) δ 7.56 (d, |
| | [1,1′;2′,1″]terphenyl-4′- | 67 | J = 1.76 Hz, 1H), 7.53 (s, 1H), 7.45 (d, |
| | ylmethyl]-amino}-propyl)- | | J = 7.33 Hz, 1H), 7.10-7.27 (m, 10H), |
| | phosphonic acid | | 6.97-7.04 (m, 4H), 4.18 (s, 2H), |
| | | | 3.12 (t, J = 6.30 Hz, 2H), 2.57 (t, J = 7.62 Hz, |
| | | | 4H), 1.82-2.07 (m, 4H), |
| | | | 1.61-1.75 (m, 2H) |
| 13 | [3-({6-(3-Chloro-phenyl)- | Intermediate | ¹H NMR (300 MHz, CD₃OD) δ 7.86 (d, |
| | 5-[4-(3-phenyl-propyl)- | 72 | J = 7.91 Hz, 1H), 7.54 (d, J = 7.91 Hz, |
| | phenyl]-pyridin-2- | | 1H), 7.38-7.40 (m, 1H), |
| | ylmethyl}-amino)-propyl]- | | 7.07-7.30 (m, 12H), 4.24 (s, 2H), 3.06 (t, J = 6.45 Hz, |
| | phosphonic acid | | 2H), 2.62 (dt, J = 7.66, 9.89 Hz, |
| | | | 4H), 1.94 (d, J = 7.33 Hz, 4H), |
| | | | 1.60-1.73 (m, 2H) |
| 14 | [3-({6-Phenyl-5-[4-(3- | Intermediate | ¹H NMR (300 MHz, CD₃OD) δ 7.84 (d, |
| | phenyl-propyl)-phenyl]- | 68 | J = 7.91 Hz, 1H), 7.52 (d, J = 7.91 Hz, |
| | pyridin-2-ylmethyl}- | | 1H), 7.34-7.39 (m, 2H), |
| | amino)-propyl]- | | 7.20-7.27 (m, 5H), 7.05-7.16 (m, 7H), 4.34 (s, |
| | phosphonic acid | | 2H), 3.17 (t, J = 6.74 Hz, 2H), |
| | | | 2.56-2.64 (m, 4H), 1.85-2.10 (m, 4H), |
| | | | 1.62-1.75 (m, 2H) |
| 15 | (3-{[6-(3-Chloro-phenyl)- | Intermediate | ¹H NMR (300 MHz CD₃OD) δ 7.87 (d, |
| | 5-(4-hexyl-3-propyl- | 71 | J = 7.91 Hz, 1H), 7.54 (d, J = 7.91 Hz, |
| | phenyl)-pyridin-2- | | 1H), 7.35-7.37 (m, 1H), 7.30 (tt, J = 1.87, |
| | ylmethyl]-amino}-propyl)- | | 6.63 Hz, 1H), 7.19-7.26 (m, |
| | phosphonic acid | | 2H), 7.12 (d, J = 7.91 Hz, 1H), |
| | | | 6.99 (dd, J = 1.90, 7.76 Hz, 1H), 6.86 (d, J = 2.05 Hz, |
| | | | 1H), 4.35 (s, 2H), 3.18 (t, J = 7.60 Hz, |
| | | | 2H), 2.61 (t, J = 8.20 Hz, |
| | | | 2H), 2.49 (t, J = 7.90 Hz, 2H), |
| | | | 1.95-2.09 (m, 2H), 1.62-1.75 (m, 4H), |
| | | | 1.50-1.61 (m, 2H), 1.29-1.48 (m, 6H), |
| | | | 0.90 (t, J = 6.40 Hz, 3H), 0.84 (t, J = 7.30 Hz, |
| | | | 3H) |
| 16 | (3-{[5-(4-Hexyl-3-propyl- | Intermediate | ¹H NMR (300 MHz, CD₃OD) δ 7.86 (d, |
| | phenyl)-6-phenyl-pyridin- | 70 | J = 7.91 Hz, 1H), 7.50 (d, J = 7.91 Hz, |
| | 2-ylmethyl]-amino}- | | 1H), 7.35-7.39 (m, 2H), |
| | propyl)-phosphonic acid | | 7.22-7.27 (m, 3H), 7.09 (d, J = 7.91 Hz, 1H), |
| | | | 6.99 (dd, J = 1.80, 7.91 Hz, 1H), |
| | | | 6.85 (d, J = 1.76 Hz, 1H), 4.35 (s, 2H), |
| | | | 3.19 (t, J = 5.86 Hz, 3H), 2.59 (t, J = 8.20 Hz, |
| | | | 2H), 2.45 (t, J = 8.20 Hz, 2H), |
| | | | 1.95-2.09 (m, 2H), 1.48-1.75 (m, |
| | | | 6H), 1.30-1.46 (m, 6H), 0.91 (t, J = 6.15 Hz, |
| | | | 3H), 0.81 (t, J = 7.33 Hz, 3H) |
| 17 | (3-{[6-(3-Fluoro-phenyl)- | Intermediate | ¹H NMR (300 MHz, CD₃OD) δ 7.84 (d, |
| | 5-(4-pentyloxy-phenyl)- | 75 | J = 7.91 Hz, 1H), 7.52 (d, J = 7.91 Hz, |
| | pyridin-2-ylmethyl]- | | 1H), 7.06-7.28 (m, 5H), |
| | amino}-propyl)- | | 6.97-7.04 (m, 1H), 6.85 (d, J = 8.79 Hz, 2H), |
| | phosphonic acid | | 4.29 (s, 2H), 3.96 (t, J = 6.45 Hz, 2H), |
| | | | 3.12 (t, J = 6.45 Hz, 2H), |
| | | | 1.92-2.08 (m, 2H), 1.60-1.82 (m, 4H), |
| | | | 1.35-1.49 (m, 4H), 0.94 (t, J = 7.03 Hz, 3H) |
| 18 | (3-{[6-(4-Fluoro-phenyl)- | Intermediate | ¹H NMR (300 MHz, CD₃OD) δ 7.82 (d, |
| | 5-(4-pentyloxy-phenyl)- | 76 | J = 7.91 Hz, 1H), 7.49 (d, J = 7.91 Hz, |
| | pyridin-2-ylmethyl]- | | 1H), 7.38-7.45 (m, 2H), |
| | amino}-propyl)- | | 6.95-7.10 (m, 4H), 6.84 (d, J = 8.79 Hz, 2H), |
| | phosphonic acid | | 4.29 (s, 2H), 3.95 (t, J = 6.45 Hz, 2H), |
| | | | 3.13 (t, J = 6.45 Hz, 2H), |
| | | | 1.93-2.08 (m, 2H), 1.60-1.81 (m, 4H), |
| | | | 1.33-1.51 (m, 4H), 0.94 (t, J = 7.00 Hz, 3H) |
| 19 | (3-{[5-(4-Pentyloxy- | Intermediate | ¹H NMR (300 MHz, CD₃OD) δ 7.83 (d, |
| | phenyl)-6-phenyl-pyridin- | 74 | J = 7.91 Hz, 1H), 7.50 (d, J = 7.91 Hz, |
| | 2-ylmethyl]-amino}- | | 1H), 7.36-7.40 (m, 2H), |
| | propyl)-phosphonic acid | | 7.23-7.28 (m, 3H), 7.03-7.08 (m, 2H), |
| | | | 6.78-6.83 (m, 2H), 4.33 (s, 2H), 3.93 (t, J = 6.45 Hz, |
| | | | 2H), 3.17 (t, J = 6.59 Hz, |
| | | | 2H), 1.94-2.09 (m, 2H), |
| | | | 1.61-1.80 (m, 4H), 1.33-1.50 (m, 4H), 0.94 (t, J = 7.00 Hz, |
| | | | 3H) |
| 20 | (3-{[6-(6-Octyl-pyridin-3- | Intermediate | ¹H NMR (300 MHz, CD₃OD) δ |
| | yl)-biphenyl-3-ylmethyl]- | 66 | 8.10 (dd, J = 0.59, 2.34 Hz, 1H), 7.63 (dd, J = 2.05, |
| | amino}-propyl)- | | 7.91 Hz, 1H), 7.59 (d, J = 1.47 Hz, |
| | phosphonic acid | | 1H), 7.50 (dd, J = 2.05, 8.20 Hz, |
| | | | 2H), 7.12-7.27 (m, 6H), 4.19 (s, 2H), |
| | | | 3.10 (t, J = 6.15 Hz, 2H), 2.72 (t, J = 7.90 Hz, |
| | | | 2H), 1.93-2.08 (m, 2H), |
| | | | 1.61-1.74 (m, 4H), 1.30 (s, 6H), |
| | | | 0.89 (t, J = 6.54 Hz, 3H) |
| 21 | {3-[(4-Octyl- | Intermediate | ¹H NMR (300 MHz, CD₃OD) δ 7.53 (s, |
| | [1,1′;2′,1″]terphenyl-4′- | 77 | 2H), 7.41-7.47 (m, 1H), |
| | ylmethyl)-amino]-propyl}- | | 7.10-7.20 (m, 5H), 6.95-7.05 (m, 4H), 4.16 (d, J = 9.38 Hz, |
| | phosphonic acid | | 2H), 3.04-3.14 (m, 2H), |
| | | | 2.54 (t, J = 7.33 Hz, 2H), |
| | | | 1.93-2.05 (m, 2H), 1.53-1.74 (m, 4H), |
| | | | 1.26-1.48 (m, 6H), 0.89 (t, J = 7.00 Hz, 3H) |
| 22 | (3-{[6-(6-Octyl-pyridin-3- | | ¹H NMR (300 MHz, CD₃OD) δ 8.09 (d, |
| | yl)-biphenyl-3-ylmethyl]- | | J = 1.76 Hz, 1H), 7.59-7.65 (m, 2H), |
| | amino}-propyl)- | | 7.47-7.52 (m, 2H), 7.11-7.26 (m, |
| | phosphonic acid | | 6H), 4.20 (s, 2H), 3.12 (t, J = 6.01 Hz, |
| | | | 2H), 2.72 (t, J = 7.62 Hz, 2H), |
| | | | 1.93-2.11 (m, 2H), 1.60-1.76 (m, 4H), |
| | | | 1.29 (d, J = 3.22 Hz, 10H), 0.89 (t, J = 6.74 Hz, |
| | | | 3H) |
| 23 | Phosphoric acid mono- | Intermediate | ¹H NMR (300 MHz, CD₃OD) δ |
| | {2-[(4-hexyl- | 62 | 7.56 (dd, J = 2.05, 7.91 Hz, 1H), 7.52 (s, |
| | [1,1′;2′,1″]terphenyl-4′- | | 1H), 7.42 (d, J = 7.62 Hz, 1H), |
| | ylmethyl)-amino]-ethyl}ester | | 7.10-7.20 (m, 5H), 6.95-7.02 (m, 4H), |
| | | | 4.21 (s, 2H), 4.07-4.14 (m, 2H), |
| | | | 3.16-3.19 (m, 2H), 2.54 (t, J = 7.60 Hz, 2H), |
| | | | 1.51-1.71 (m, 2H), 1.26-1.33 (m, |
| | | | 6H), 0.89 (t, J = 6.74 Hz, 3H) |
| 25 | [1-(4-Hexyl- | Intermediate | ¹H NMR (300 MHz, CD₃OD) δ |
| | [1,1′;2′,1″]terphenyl-4′- | 62 | 7.35-7.44 (m, 3H), 7.10-7.19 (m, 5H), |
| | ylmethyl)-pyrrolidin-3-yl]- | | 7.00 (s, 4H), 3.79-3.91 (m, 2H), |
| | phosphonic acid | | 3.14-3.23 (m, 1H), 2.97-3.04 (m, 1H), |
| | | | 2.61-2.78 (m, 2H), 2.55 (t, J = 7.62 Hz, |
| | | | 2H), 2.30-2.44 (m, 1H), |
| | | | 2.06-2.19 (m, 2H), 1.52-1.61 (m, 2H), 1.30 (s, |
| | | | 6H), 0.89 (t, J = 6.45 Hz, 3H) |
| 26 | [1-(4-Hexyl- | Intermediate | ¹H NMR (300 MHz, CD₃OD) δ |
| | [1,1′;2′,1″]terphenyl-4′- | 62 | 7.40-7.49 (m, 3H), 7.16-7.21 (m, 3H), |
| | ylmethyl)-pyrrolidin-3-yl]- | | 7.10-7.14 (m, 2H), 7.00 (s, 4H), 4.09 (s, |
| | phosphonic acid | | 2H), 3.92 (quin, J = 7.00 Hz, 2H), |
| | monoethyl ester | | 3.35 (s, 1H), 3.14-3.23 (m, 1H), |
| | | | 2.90-3.02 (m, 2H), 2.39-2.58 (m, 3H), |
| | | | 2.10-2.23 (m, 2H), 1.52-1.61 (m, 2H), |
| | | | 1.27-1.34 (m, 6H), 1.23 (t, J = 7.03 Hz, |
| | | | 3H), 0.88 (t, J = 7.00 Hz, 3H) |
| 27 | (3-{[5-(4-Hexyl-phenyl)-6- | Intermediate | ¹H NMR (300 MHz, CD₃OD) δ 7.85 (d, |
| | phenyl-pyridin-2- | 65 | J = 7.91 Hz, 1H), 7.51 (d, J = 7.91 Hz, |
| | ylmethyl]-amino}-propyl)- | | 1H), 7.35-7.38 (m, 2H), |
| | phosphonic acid | | 7.19-7.27 (m, 3H), 7.04-7.11 (m, 4H), 4.32 (s, |
| | | | 2H), 3.15 (t, J = 6.59 Hz, 2H), 2.59 (t, |
| | | | J = 7.62 Hz, 2H), 1.94-2.09 (m, 2H), |
| | | | 1.54-1.75 (m, 4H), 1.27-1.48 (m, |
| | | | 6H), 0.89 (t, J = 6.45 Hz, 3H) |
| 28 | (3-{[(4-hexyl-1,1′:2′,1″- | Intermediate | ¹H NMR (300 MHz, CD₃OD) δ |
| | terphenyl-4′- | 62 | 7.47-7.70 (m, 2H), 7.42 (d, J = 7.92 Hz, |
| | yl)methyl]amino}propyl)phosphonic | | 1H), 7.05-7.28 (m, 5H), |
| | acid | | 6.88-7.05 (m, 4H), 4.17 (s, 2H), 3.11 (t, J = 6.45 Hz, |
| | | | 2H), 2.53 (t, J = 7.62 Hz, 2H), |
| | | | 1.86-2.10 (m, 2H), 1.46-1.79 (m, |
| | | | 4H), 1.16-1.41 (m, 6H), |
| | | | 0.70-0.95 (m, 3H). |

### Example 16

### Biological Data

Novel compounds were synthesized and tested for S1P1 activity using the GTP γ³⁵S binding assay. These compounds may be assessed for their ability to activate or block activation of the human S1P1 receptor in cells stably expressing the S1P1 receptor. GTP γ³⁵S binding was measured in the medium containing (mM) HEPES 25, pH 7.4, MgCl₂ 10, NaCl 100, dithitothreitol 0.5, digitonin 0.003%, 0.2 nM GTP γ³⁵S, and 5 μg membrane protein in a volume of 150 μl. Test compounds were included in the concentration range from 0.08 to 5,000 nM unless indicated otherwise. Membranes were incubated with 100 μM 5′-adenylylimmidodiphosphate for 30 min, and subsequently with 10 μM GDP for 10 min on ice. Drug solutions and membrane were mixed, and then reactions were initiated by adding GTP γ³⁵S and continued for 30 min at 25° C. Reaction mixtures were filtered over Whatman GF/B filters under vacuum, and washed three times with 3 mL of ice-cold buffer (HEPES 25, pH7.4, MgCl₂ 10 and NaCl 100). Filters were dried and mixed with scintillant, and counted for ³⁵S activity using a β-counter. Agonist-induced GTP γ³⁵S binding was obtained by subtracting that in the absence of agonist. Binding data were analyzed using a non-linear regression method. In case of antagonist assay, the reaction mixture contained 10 nM S1P in the presence of test antagonist at concentrations ranging from 0.08 to 5000 nM.

Table 8 shows activity potency: S1P1 receptor from GTP γ³⁵S: nM, (EC₅₀), and stimulation (%).

Activity potency: S1P1 receptor from GTP γ³⁵S: nM, (EC₅₀)

TABLE 8

| | | % |
|---|---|---|
| | | STIMU- |
| | | LATION |
| Compound | GTPγ³⁵S | @ 5 μM |
| IUPAC name | EC50 (nM) | (%) |
| (3-{[6-(5-Hexyl-pyridin-2-yl)-biphenyl-3- | 316.74 | 79.40 |
| ylmethyl]-amino}-propyl)-phosphonic acid | | |
| (3-{[6-(6-Hexyl-pyridin-3-yl)-biphenyl-3- | 833.01 | 94.70 |
| ylmethyl]-amino}-propyl)-phosphonic acid | | |
| (3-{[5-(4-Hexyl-phenyl)-6-phenyl-pyridin-2- | 252.03 | 112.30 |
| ylmethyl]-amino}-propyl)-phosphonic acid | | |
| (3-{[6-(6-Octyl-pyridin-3-yl)-biphenyl-3- | 771.47 | 89.70 |
| ylmethyl]-amino}-propyl)-phosphonic acid | | |
| (3-{[5-(4-Pentyloxy-phenyl)-6-phenyl- | 357.84 | 95.50 |
| pyridin-2-ylmethyl]-amino}-propyl)- | | |
| phosphonic acid | | |
| (3-{[6-(4-Fluoro-phenyl)-5-(4-pentyloxy- | 472.12 | 104.20 |
| phenyl)-pyridin-2-ylmethyl]-amino}-propyl)- | | |
| phosphonic acid | | |
| (3-{[6-(3-Fluoro-phenyl)-5-(4-pentyloxy- | 2200.43 | 83.90 |
| phenyl)-pyridin-2-ylmethyl]-amino}-propyl)- | | |
| phosphonic acid | | |
| (3-{[4-(3-Phenyl-propyl)- | 625.06 | 64.00 |
| [1,1′;2′,1″]terphenyl-4′-ylmethyl]-amino}- | | |
| propyl)-phosphonic acid | | |
| (3-{[4-(3,3,4,4,5,5,6,6,6-Nonafluoro-hexyl)- | 416.06 | 71.80 |
| [1,1′;2′,1″]terphenyl-4′-ylmethyl]-amino}- | | |
| propyl)-phosphonic acid | | |
| (3-{[5-(4-Hexyl-3-propyl-phenyl)-6-phenyl- | 29.59 | 73.10 |
| pyridin-2-ylmethyl]-amino}-propyl)- | | |
| phosphonic acid | | |
| [3-({6-Phenyl-5-[4-(3-phenyl-propyl)- | 93.84 | 86.60 |
| phenyl]-pyridin-2-ylmethyl}-amino)-propyl]- | | |
| phosphonic acid | | |
| [3-({6-(3-Chloro-phenyl)-5-[4-(3-phenyl- | 2427.07 | 64.00 |
| propyl)-phenyl]-pyridin-2-ylmethyl}-amino)- | | |
| propyl]-phosphonic acid | | |
| (3-{[5-(4-Hexyl-phenyl)-6-thiophen-2-yl- | 5.26 | 68.10 |
| pyridin-2-ylmethyl]-amino}-propyl)- | | |
| phosphonic acid | | |
| (3-{[5-(4-Hexyl-phenyl)-6-thiophen-3-yl- | 9.19 | 76.90 |
| pyridin-2-ylmethyl]-amino}-propyl)- | | |
| phosphonic acid | | |
| (3-{[6-Furan-2-yl-5-(4-hexyl-phenyl)-pyridin- | 0.44 | 88.60 |
| 2-ylmethyl]-amino}-propyl)-phosphonic acid | | |
| (3-{[5-(4-Hexyl-phenyl)-6-oxazol-4-yl- | 0.87 | 86.10 |
| pyridin-2-ylmethyl]-amino}-propyl)- | | |
| phosphonic acid | | |
| (3-{[5-(4-Hexyl-phenyl)-6-thiazol-2-yl- | 6.52 | 89.30 |
| pyridin-2-ylmethyl]-amino}-propyl)- | | |
| phosphonic acid | | |
| [3-({5-[4-(3-Phenyl-propyl)-phenyl]-6- | 2.36 | 91.10 |
| thiophen-2-yl-pyridin-2-ylmethyl}-amino)- | | |
| propyl]-phosphonic acid | | |
| (3-{[5-(4-Hexyl-3-propyl-phenyl)-6-thiophen- | 7.72 | 79.10 |
| 2-yl-pyridin-2-ylmethyl]-amino}-propyl)- | | |
| phosphonic acid | | |
| (3-{[6-(5-Fluoro-thiophen-2-yl)-5-(4-hexyl- | 25.05 | 110.70 |
| phenyl)-pyridin-2-ylmethyl]-amino}-propyl)- | | |
| phosphonic acid | | |

What is claimed is:

1. A compound having Formula I, its individual enantiomers, individual diastereoisomers, and individual isomers, individual tautomers or a pharmaceutically acceptable salt thereof 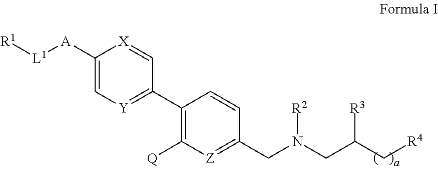
wherein:
R¹ is Me, CF₃ or aryl;
R² is H, C₁₋₁₀ alkyl, or together with R³ forms a 5 or 6 membered heterocycle ring;
R³ is H, C₁₋₁₀ alkyl, or together with R² forms a 5 or 6 membered heterocycle ring;
R⁴ is OPO₃H₂, carboxylic acid, C₁₋₆ alkyl, —S(O)₂H, —P(O)(OH)(OR¹⁰), —P(O)(H)OH or OR⁹;
X is CR⁵ or N;
Y is CR⁶ or N;
Z is CR⁷ or N;
A is O, CH₂ or NR⁸;
L¹ is C₂₋₁₀ alkylene;
R⁵ is H, C₁₋₁₀ alkyl, C₂₋₆ alkenyl or C₃₋₁₀ cycloalkyl;
R⁶ is H, C₁₋₁₀ alkyl, C₂₋₆ alkenyl or C₃₋₁₀ cycloalkyl;
R⁷ is H, C₁₋₁₀ alkyl, C₂₋₆ alkenyl or C₃₋₁₀ cycloalkyl;
R⁸ is H, C₃₋₁₀ cycloalkyl or C₁₋₆ alkyl;
R⁹ is H or C₁₋₁₀ alkyl;
R¹⁰ is H or C₁₋₁₀ alkyl;
Q is C₃₋₁₀ cycloalkyl, heterocycle or aryl; and
a is 0, 1, 2, 3 or 4.

2. A compound according to claim 1 wherein:
R¹ is Me, CF₃, phenyl;
R² is H, or together with R³ forms a 5 membered heterocycle ring;
R³ is H, or together with R² forms a 5 membered heterocycle ring;
R⁴ is —P(O)(OH)(OR¹⁰);
X is CR⁵ or N;
Y is CR⁶ or N;
Z is CR⁷ or N;
A is O or CH₂;
L¹ is C₂₋₅ alkylene;
R⁵ is H or C₁₋₆ alkyl;
R⁶ is H;
R⁷ is H;
R¹⁰ is H or C₁₋₆ alkyl;
Q is heterocycle or aryl; and
a is 0 or 1.

3. A compound according to claim 2 wherein:
R¹ is Me or phenyl;
R² is H;
R³ is H;
R⁴ is —P(O)(OH)(OR¹⁰);
X is CR⁵;
Y is CR⁶ or N;
Z is N;
A is CH₂;
L¹ is C₂₋₅ alkylene;
R⁵ is H or C₁₋₆ alkyl;
R⁶ is H;
R¹⁰ is H;
Q is heterocycle or aryl; and
a is 1.

4. A compound according to claim 1 selected from:
(3-{[6-(5-Hexyl-pyridin-2-yl)-biphenyl-3-ylmethyl]-amino}-propyl)-phosphonic acid;
(3-{[6-(6-Hexyl-pyridin-3-yl)-biphenyl-3-ylmethyl]-amino}-propyl)-phosphonic acid;
(3-{[5-(4-Hexyl-phenyl)-6-phenyl-pyridin-2-ylmethyl]-amino}-propyl)-phosphonic acid;
[1-(4-Hexyl-[1,1′;2′,1″]terphenyl-4′-ylmethyl)-pyrrolidin-3-yl]-phosphonic acid monoethyl ester;
[1-(4-Hexyl-[1,1′;2′,1″]terphenyl-4′-ylmethyl)-pyrrolidin-3-yl]-phosphonic acid;
(3-{[6-(6-Octyl-pyridin-3-yl)-biphenyl-3-ylmethyl]-amino}-propyl)-phosphonic acid;
(3-{[5-(4-Pentyloxy-phenyl)-6-phenyl-pyridin-2-ylmethyl]-amino}-propyl)-phosphonic acid;
(3-{[6-(4-Fluoro-phenyl)-5-(4-pentyloxy-phenyl)-pyridin-2-ylmethyl]-amino}-propyl)-phosphonic acid;
(3-{[6-(3-Fluoro-phenyl)-5-(4-pentyloxy-phenyl)-pyridin-2-ylmethyl]-amino}-propyl)-phosphonic acid;
(3-{[4-(3-Phenyl-propyl)[1,1′;2′,1″]terphenyl-4′-ylmethyl]-amino}-propyl)-phosphonic acid;
(3-{[4-(3,3,4,4,5,5,6,6,6-Nonafluoro-hexyl)-[1,1′;2′,1″]terphenyl-4′-ylmethyl]-amino}-propyl)-phosphonic acid;
(3-{[5-(4-Hexyl-3-propyl-phenyl)-6-phenyl-pyridin-2-ylmethyl]-amino}-propyl)-phosphonic acid;
(3-{[6-(3-Chloro-phenyl)-5-(4-hexyl-3-propyl-phenyl)-pyridin-2-ylmethyl]-amino}-propyl)-phosphonic acid;
[3-({6-Phenyl-5-[4-(3-phenyl-propyl)-phenyl]-pyridin-2-ylmethyl}-amino)-propyl]-phosphonic acid;
[3-({6-(3-Chloro-phenyl)-5-[4-(3-phenyl-propyl)-phenyl]-pyridin-2-ylmethyl}-amino)-propyl]-phosphonic acid;
(3-{[5-(4-Hexyl-phenyl)-6-(3-hydroxy-phenyl)-pyridin-2-ylmethyl]-amino}-propyl)-phosphonic acid;
(3-{[5-(4-Hexyl-phenyl)-6-thiophen-2-yl-pyridin-2-ylmethyl]-amino}-propyl)-phosphonic acid;
(3-{[5-(4-Hexyl-phenyl)-6-thiophen-3-yl-pyridin-2-ylmethyl]-amino}-propyl)-phosphonic acid;
(3-{[6-Furan-2-yl-5-(4-hexyl-phenyl)-pyridin-2-ylmethyl]-amino}-propyl)-phosphonic acid;
(3-{[5-(4-Hexyl-phenyl)-6-oxazol-4-yl-pyridin-2-ylmethyl]-amino}-propyl)-phosphonic acid;
(3-{[5-(4-Hexyl-phenyl)-6-thiazol-2-yl-pyridin-2-ylmethyl]-amino}-propyl)-phosphonic acid;
[3-({5-[4-(3-Phenyl-propyl)-phenyl]-6-thiophen-2-yl-pyridin-2-ylmethyl}-amino)-propyl]-phosphonic acid;
(3-{[5-(4-Hexyl-3-propyl-phenyl)-6-thiophen-2-yl-pyridin-2-ylmethyl]-amino}-propyl)-phosphonic acid;
(3-{[3-(4-Hexyl-phenyl)-[2,3′]bipyridinyl-6-ylmethyl]-amino}-propyl)-phosphonic acid;
(3-{[6-(5-Fluoro-thiophen-2-yl)-5-(4-hexyl-phenyl)-pyridin-2-ylmethyl]-amino}-propyl)-phosphonic acid.

5. A compound according to claim 1 selected from:
(3-{[5-(4-Hexyl-3-propyl-phenyl)-6-phenyl-pyridin-2-ylmethyl]-amino}-propyl)-phosphonic acid;
[3-({6-Phenyl-5-[4-(3-phenyl-propyl)-phenyl]-pyridin-2-ylmethyl}-amino)-propyl]-phosphonic acid;
(3-{[5-(4-Hexyl-phenyl)-6-thiophen-2-yl-pyridin-2-ylmethyl]-amino}-propyl)-phosphonic acid;
(3-{[5-(4-Hexyl-phenyl)-6-thiophen-3-yl-pyridin-2-ylmethyl]-amino}-propyl)-phosphonic acid;
(3-{[6-Furan-2-yl-5-(4-hexyl-phenyl)-pyridin-2-ylmethyl]-amino}-propyl)-phosphonic acid;
(3-{[5-(4-Hexyl-phenyl)-6-oxazol-4-yl-pyridin-2-ylmethyl]-amino}-propyl)-phosphonic acid;
(3-{[5-(4-Hexyl-phenyl)-6-thiazol-2-yl-pyridin-2-ylmethyl]-amino}-propyl)-phosphonic acid;
[3-({5-[4-(3-Phenyl-propyl)-phenyl]-6-thiophen-2-yl-pyridin-2-ylmethyl}-amino)-propyl]-phosphonic acid;
(3-{[5-(4-Hexyl-3-propyl-phenyl)-6-thiophen-2-yl-pyridin-2-ylmethyl]-amino}-propyl)-phosphonic acid;
(3-{[6-(5-Fluoro-thiophen-2-yl)-5-(4-hexyl-phenyl)-pyridin-2-ylmethyl]-amino}-propyl)-phosphonic acid.

6. A pharmaceutical composition comprising as active ingredient a therapeutically effective amount of a compound according to claim 1 and a pharmaceutically acceptable adjuvant, diluents or carrier.

7. A pharmaceutical composition according to claim 6 wherein the compound is selected from:
(3-{[6-(5-Hexyl-pyridin-2-yl)-biphenyl-3-ylmethyl]-amino}-propyl)-phosphonic acid;
(3-{[6-(6-Hexyl-pyridin-3-yl)-biphenyl-3-ylmethyl]-amino}-propyl)-phosphonic acid;
(3-{[5-(4-Hexyl-phenyl)-6-phenyl-pyridin-2-ylmethyl]-amino}-propyl)-phosphonic acid;
[1-(4-Hexyl-[1,1′;2′,1″]terphenyl-4′-ylmethyl)-pyrrolidin-3-yl]-phosphonic acid monoethyl ester;
[1-(4-Hexyl-[1,1′;2′,1″]terphenyl-4′-ylmethyl)-pyrrolidin-3-yl]-phosphonic acid;
(3-{[6-(6-Octyl-pyridin-3-yl)-biphenyl-3-ylmethyl]-amino}-propyl)-phosphonic acid;
(3-{[5-(4-Pentyloxy-phenyl)-6-phenyl-pyridin-2-ylmethyl]-amino}-propyl)-phosphonic acid;
(3-{[6-(4-Fluoro-phenyl)-5-(4-pentyloxy-phenyl)-pyridin-2-ylmethyl]-amino}-propyl)phosphonic acid;
(3-{[6-(3-Fluoro-phenyl)-5-(4-pentyloxy-phenyl)-pyridin-2-ylmethyl]-amino}-propyl)-phosphonic acid;
(3-{[4-(3-Phenyl-propyl)[1,1′;2′,1″]terphenyl-4′-ylmethyl]-amino}-propyl)-phosphonic acid;
(3-{[4-(3,3,4,4,5,5,6,6,6-Nonafluoro-hexyl)-[1,1′;2′,1″]terphenyl-4′-ylmethyl]-amino}-propyl)-phosphonic acid;
(3-{[5-(4-Hexyl-3-propyl-phenyl)-6-phenyl-pyridin-2-ylmethyl]-amino}-propyl)-phosphonic acid;
(3-{[6-(3-Chloro-phenyl)-5-(4-hexyl-3-propyl-phenyl)-pyridin-2-ylmethyl]-amino}-propyl)-phosphonic acid;
[3-({6-Phenyl-5-[4-(3-phenyl-propyl)-phenyl]-pyridin-2-ylmethyl}-amino)-propyl]-phosphonic acid;
[3-({6-(3-Chloro-phenyl)-5-[4-(3-phenyl-propyl)-phenyl]-pyridin-2-ylmethyl}-amino)-propyl]-phosphonic acid;
(3-{[5-(4-Hexyl-phenyl)-6-(3-hydroxy-phenyl)-pyridin-2-ylmethyl]-amino}-propyl)-phosphonic acid;
(3-{[5-(4-Hexyl-phenyl)-6-thiophen-2-yl-pyridin-2-ylmethyl]-amino}-propyl)-phosphonic acid;
(3-{[5-(4-Hexyl-phenyl)-6-thiophen-3-yl-pyridin-2-ylmethyl]-amino}-propyl)-phosphonic acid;
(3-{[6-Furan-2-yl-5-(4-hexyl-phenyl)-pyridin-2-ylmethyl]-amino}-propyl)-phosphonic acid;
(3-{[5-(4-Hexyl-phenyl)-6-oxazol-4-yl-pyridin-2-ylmethyl]-amino}-propyl)-phosphonic acid;
(3-{[5-(4-Hexyl-phenyl)-6-thiazol-2-yl-pyridin-2-ylmethyl]-amino}-propyl)-phosphonic acid;
[3-({5-[4-(3-Phenyl-propyl)-phenyl]-6-thiophen-2-yl-pyridin-2-ylmethyl}-amino)-propyl]-phosphonic acid;
(3-{[5-(4-Hexyl-3-propyl-phenyl)-6-thiophen-2-yl-pyridin-2-ylmethyl]-amino}-propyl)-phosphonic acid;
(3-{[3-(4-Hexyl-phenyl)-[2,3′]bipyridinyl-6-ylmethyl]-amino}-propyl)-phosphonic acid;
(3-{[6-(5-Fluoro-thiophen-2-yl)-5-(4-hexyl-phenyl)-pyridin-2-ylmethyl]-amino}-propyl)-phosphonic acid.

8. A method of treating a disorder associated with sphingosine-1-phosphate receptor modulation, which comprises administering to a mammal in need thereof, a pharmaceutical composition comprising a therapeutically effective amount of at least one compound of Formula I 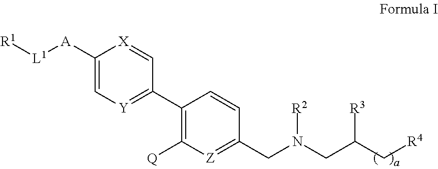
wherein:
R¹ is Me, CF₃ or aryl;
R² is H, C₁₋₁₀ alkyl, or together with R³ forms a 5 or 6 membered heterocycle ring;
R³ is H, C₁₋₁₀ alkyl, or together with R² forms a 5 or 6 membered heterocycle ring;
R⁴ is OPO₃H₂, carboxylic acid, C₁₋₆ alkyl, —S(O)₂H, —P(O)(OH)(OR¹⁰), —P(O)(H)OH or OR⁹;
X is CR⁵ or N;
Y is CR⁶ or N;
Z is CR⁷ or N;
A is O, CH₂ or NR⁸;
L¹ is C₂₋₁₀ alkylene;
R⁵ is H, C₁₋₁₀ alkyl, C₂₋₆ alkenyl or C₃₋₁₀ cycloalkyl;
R⁶ is H, C₁₋₁₀ alkyl, C₂₋₆ alkenyl or C₃₋₁₀ cycloalkyl;
R⁷ is H, C₁₋₁₀ alkyl, C₂₋₆ alkenyl or C₃₋₁₀ cycloalkyl;
R⁸ is H, C₃₋₁₀ cycloalkyl or C₁₋₆ alkyl;
R⁹ is H or C₁₋₁₀ alkyl;
R¹⁰ is H or C₁₋₁₀ alkyl;
Q is C₃₋₁₀ cycloalkyl, heterocycle or aryl; and
a is 0, 1, 2, 3 or 4.

9. A method of claim 8, wherein the pharmaceutical composition is administered to the mammal to treat ocular diseases, wet and dry age-related macular degeneration, diabetic retinopathy, retinopathy of prematurity, retinal edema, geographic atrophy, glaucomatous optic neuropathy, chorioretinopathy, hypertensive retinopathy, ocular ischemic syndrome, prevention of inflammation-induced fibrosis in the back of the eye, various ocular inflammatory diseases including uveitis, scleritis, keratitis, and retinal vasculitis; or systemic vascular barrier related diseases, various inflammatory diseases, including acute lung injury, its prevention, sepsis, tumor metastasis, atherosclerosis, pulmonary edemas, and ventilation-induced lung injury; or autoimmune diseases and immunosuppression, rheumatoid arthritis, Crohn's disease, Graves' disease, inflammatory bowel disease, multiple sclerosis, Myasthenia gravis, Psoriasis, ulcerative colitis, antoimmune uveitis, renal ischemia perfusion injury, contact hypersensitivity, atopic dermititis, and organ transplantation; or allergies and other inflammatory diseases, urticaria, bronchial asthma, and other airway inflammations including pulmonary emphysema and chronic obstructive pulmonary diseases; or cardiac protection, ischemia reperfusion injury and atherosclerosis; or wound healing such as but not limited to: scar-free healing of wounds from cosmetic skin surgery, ocular surgery, GI surgery, general surgery, oral injuries, various mechanical, heat and burn injuries, prevention and treatment of photoaging and skin ageing, and prevention of radiation-induced injuries; or bone formation, treatment of osteoporosis and various bone fractures including hip and ankles; or anti-nociceptive activity, visceral pain, pain associated with diabetic neuropathy, rheumatoid arthritis, chronic knee and joint pain, tendonitis, osteoarthritis, neuropathic pains; or central nervous system neuronal activity in Alzheimer's disease, age-related neuronal injuries; or organ transplant such as renal, corneal, cardiac or adipose tissue transplant.

10. The method of claim 9 wherein the mammal is a human.
